SEQUENCE LISTING (64 sequences)

SEQ ID NO: 1 - protein, 32 aa, unidentified
  source 1..32: note = organism=AdV5
ATPSMMPQWSYMHISGQDASEYLSPGLVQFAR

SEQ ID NO: 2 - protein, 32 aa, unidentified; identical to SEQ ID NO: 1
  source 1..32: note = organism=AdV5

SEQ ID NO: 3 - protein, 32 aa, unidentified; identical to SEQ ID NO: 1
  source 1..32: note = organism=AdV5

SEQ ID NO: 4 - protein, 24 aa, unidentified
  source 1..24: note = organism=AdV5
VVLYSEDVDIETPDTHISYMPTIK

SEQ ID NO: 5 - protein, 17 aa, unidentified
  source 1..17: note = organism=AdV5
VGNNFAMEINLNANLWR

SEQ ID NO: 6 - protein, 11 aa, unidentified
  source 1..11: note = organism=AdV5
ATETYFSLNNK

SEQ ID NO: 7 - protein, 26 aa, unidentified
  source 1..26: note = organism=AdV5
TTPMKPCYGSYAKPTNENGGQGILVK

SEQ ID NO: 8 - protein, 29 aa, unidentified
  source 1..29: note = organism=AdV5
IIENHGTEDELPNYCFPLGGVINTETLTK

SEQ ID NO: 9 - protein, 9 aa, unidentified
  source 1..9: note = organism=AdV5
TGQENGWEK

SEQ ID NO: 10 - protein, 17 aa, unidentified
  source 1..17: note = organism=AdV5
VGNNFAMEINLNANLWR

SEQ ID NO: 11 - protein, 16 aa, unidentified
  source 1..16: note = organism=AdV5
WSLDYMDNVNPFNHHR

SEQ ID NO: 12 - protein, 8 aa, unidentified
  source 1..8: note = organism=AdV5
SMLLGNGR

SEQ ID NO: 13 - protein, 27 aa, unidentified
  source 1..27: note = organism=AdV5
YKDYQQVGILHQHNNSGFVGYLAPTMR

SEQ ID NO: 14 - protein, 32 aa, unidentified; identical to SEQ ID NO: 1
  source 1..32: note = organism=AdV5

SEQ ID NO: 15 - protein, 12 aa, unidentified
  source 1..12: note = organism=AdV5
QNGVLESDIGVK

SEQ ID NO: 16 - protein, 14 aa, unidentified
  source 1..14: note = organism=AdV5
SFYNDQAVYSQLIR

SEQ ID NO: 17 - protein, 8 aa, unidentified
  source 1..8: note = organism=AdV5
NSIGGVQR

SEQ ID NO: 18 - protein, 47 aa, unidentified
  source 1..47: note = organism=AdV5
ARPSEDTFNPVYPYDTETGPPTVPFLTPPFVSPNGFQESPPGVLSLR

SEQ ID NO: 19 - protein, 15 aa, unidentified
  source 1..15: note = organism=AdV5
LSEPLVTSNGMLALK

SEQ ID NO: 20 - protein, 27 aa, unidentified
  source 1..27: note = organism=AdV5
MGNGLSLDEAGNLTSQNVTTVSPPLKK

SEQ ID NO: 21 - protein, 9 aa, unidentified
  source 1..9: note = organism=AdV5
EPIYTQNGK

SEQ ID NO: 22 - protein, 32 aa, unidentified
  source 1..32: note = organism=AdV5
YGAPLHVTDDLNTLTVATGPGVTINNTSLQTK

SEQ ID NO: 23 - protein, 25 aa, unidentified
  source 1..25: note = organism=AdV5
NGDLTEGTAYTNAVGFMPNLSAYPK

SEQ ID NO: 24 - protein, 13 aa, unidentified
  source 1..13: note = organism=AdV5
SNIVSQVYLNGDK

SEQ ID NO: 25 - protein, 49 aa, unidentified
  source 1..49: note = organism=AdV5
TKSNINLEISAPLTVTSEALTVAAAAPLMVAGNTLTMQSQAPLTVHDSK

SEQ ID NO: 26 - protein, 11 aa, unidentified
  source 1..11: note = organism=AdV5
MMQDATDPAVR

SEQ ID NO: 27 - protein, 10 aa, unidentified
  source 1..10: note = organism=AdV5
MQDATDPAVR

SEQ ID NO: 28 - protein, 24 aa, unidentified
  source 1..24: note = organism=AdV5
LMVTETPQSEVYQSGPDYFFQTSR

SEQ ID NO: 29 - protein, 17 aa, unidentified
  source 1..17: note = organism=AdV5
AALQSQPSGLNSTDDWR

SEQ ID NO: 30 - protein, 17 aa, unidentified
  source 1..17: note = organism=AdV5
LLGEEEYLNNSLLQPQR

SEQ ID NO: 31 - protein, 17 aa, unidentified
  source 1..17: note = organism=AdV5
NLPPAFPNNGIESLVDK

SEQ ID NO: 32 - protein, 17 aa, unidentified
  source 1..17: note = organism=AdV5
NLPPAFPNNGIESLVDK

SEQ ID NO: 33 - protein, 14 aa, unidentified
  source 1..14: note = organism=AdV5
RPSSLSDLGAAAPR

SEQ ID NO: 34 - protein, 12 aa, unidentified
  source 1..12: note = organism=AdV5
MEDINFASLAPR

SEQ ID NO: 35 - protein, 12 aa, unidentified
  source 1..12: note = organism=AdV5
MEDINFASLAPR

SEQ ID NO: 36 - protein, 11 aa, unidentified
  source 1..11: note = organism=AdV5
AWNSSTGQMLR

SEQ ID NO: 37 - protein, 11 aa, unidentified
  source 1..11: note = organism=AdV5
AWNSSTGQMLR

SEQ ID NO: 38 - protein, 29 aa, unidentified
  source 1..29: note = organism=AdV5
SKEIPTPYMWSYQPQMGLAAGAAQDYSTR

SEQ ID NO: 39 - protein, 27 aa, unidentified
  source 1..27: note = organism=AdV5
EIPTPYMWSYQPQMGLAAGAAQDYSTR

SEQ ID NO: 40 - protein, 13 aa, unidentified
  source 1..13: note = organism=AdV5
INYMSAGPHMISR

SEQ ID NO: 41 - protein, 6 aa, unidentified
  source 1..6: note = organism=AdV5
NNLNPR

SEQ ID NO: 42 - protein, 14 aa, unidentified
  source 1..14: note = organism=AdV5
VRSPGQGITHLTIR

SEQ ID NO: 43 - protein, 17 aa, unidentified
  source 1..17: note = organism=AdV5
STNSFDGSIVSSYLTTR

SEQ ID NO: 44 - protein, 47 aa, unidentified
  source 1..47: note = organism=AdV5
QNVMGSSIDGRPVLPANSTTLTYETVSGTPLETAASAAASAAAATAR

SEQ ID NO: 45 - protein, 47 aa, unidentified; identical to SEQ ID NO: 44
  source 1..47: note = organism=AdV5

SEQ ID NO: 46 - protein, 17 aa, unidentified
  source 1..17: note = organism=AdV5
STNSFDGSIVSSYLTTR

SEQ ID NO: 47 - protein, 37 aa, unidentified
  source 1..37: note = organism=AdV5
HKDMLALPLDEGNPTPSLKPVTLQQVLPALAPSEEKR

SEQ ID NO: 48 - protein, 15 aa, unidentified
  source 1..15: note = organism=AdV5
ESGDLAPTVQLMVPK

SEQ ID NO: 49 - protein, 55 aa, unidentified
  source 1..55: note = organism=AdV5
QVAPGLGVQTVDVQIPTTSSTSIATATEGMETQTSPVASAVADAAVQAVAAAASK

SEQ ID NO: 50 - protein, 55 aa, unidentified; identical to SEQ ID NO: 49
  source 1..55: note = organism=AdV5

SEQ ID NO: 51 - protein, 15 aa, unidentified
  source 1..15: note = organism=AdV5
SILISPSNNTGWGLR

SEQ ID NO: 52 - protein, 21 aa, unidentified
  source 1..21: note = organism=AdV5
NYTPTPPPVSTVDAAIQTVVR

SEQ ID NO: 53 - protein, 21 aa, unidentified
  source 1..21: note = organism=AdV5
NYTPTPPPVSTVDAAIQTVVR

SEQ ID NO: 54 - protein, 33 aa, unidentified
  source 1..33: note = organism=AdV5
MRGGILPLLIPLIAAAIGAVPGIASVALQAQRH

SEQ ID NO: 55 - protein, 5 aa, unidentified
  source 1..5: note = organism=Anc80 AAV
ANQQK

SEQ ID NO: 56 - protein, 5 aa, unidentified
  source 1..5: note = organism=Anc80 AAV
TAPGK

SEQ ID NO: 57 - protein, 6 aa, unidentified
  source 1..6: note = organism=Anc80 AAV
QQRVSK

SEQ ID NO: 58 - protein, 5 aa, unidentified
  source 1..5: note = organism=Anc80 AAV
DGYLP

SEQ ID NO: 59 - protein, 4 aa, unidentified
  source 1..4: note = organism=Anc80 AAV
DWLE

SEQ ID NO: 60 - protein, 4 aa, unidentified
  source 1..4: note = organism=Anc80 AAV
DFAV

SEQ ID NO: 61 - protein, 15 aa, unidentified
  source 1..15: note = organism= Anc80 AAV
APGKKRPVEQSPQEP

SEQ ID NO: 62 - protein, 15 aa, unidentified
  source 1..15: note = organism=Anc80 AAV
APGKKRPVEQSPQEP

SEQ ID NO: 63 - protein, 15 aa, unidentified
  source 1..15: note = organism=Anc80 AAV
APGKKRPVEQSPQEP

SEQ ID NO: 64 - protein, 16 aa, unidentified
  source 1..16: note = organism=Anc80 AAV
TAPGKKRPVEQSPQEP

(19) United States
(12) Patent Application Publication
(10) Pub. No.: US 2025/0282833 A1
(43) Pub. Date: Sep. 11, 2025

(54) METHODS FOR PROCESSING AND ANALYZING VIRUS CAPSID PROTEINS

(71) Applicants: LONZA LTD., Visp (CH); LONZA HOUSTON, INC., Pearland, TX (US)

(72) Inventors: Mostafa ZAREI, Basel (CH); Michael JAHN, Basel (CH); Atanas KOULOV, Basel (CH); Peng WANG, Pearland, TX (US); Friedrich Michael HALLER, Pearland, TX (US); Jerome JONVEAUX, Basel (CH)

(21) Appl. No.: 18/700,957

(22) PCT Filed: Oct. 14, 2022
(86) PCT No.: PCT/EP2022/078725

§ 371 Date: Apr. 12, 2024

Related U.S. Application Data
(60) Provisional application No. 63/256,111, filed on Oct. 15, 2021.
(60) Provisional application No. 63/368,803, filed on Jul. 19, 2022.

(30) Foreign Application Priority Data
Oct. 29, 2021 (EP) 21205732.7

(51) Int. Cl. C07K 14/005; G01N 33/68

(52) U.S. Cl. CPC C07K 14/005; G01N 33/6848; C12N 2710/10022; C12N 2710/10051; C12N 2740/15022; C12N 2740/15051; C12N 2750/14122; C12N 2750/14151; G01N 2333/015; G01N 2333/075; G01N 2333/155

(57) ABSTRACT

The present disclosure provides methods for preparing digested virus proteins, including adenovirus and adeno-associated virus capsid proteins, from a sample of virus proteins, as well as methods of analyzing such digested virus proteins via liquid chromatography-tandem mass spectrometry. The methods include the use of a mixture of sodium deoxycholate (SDC) and N-dodecyl-beta-D-Maltoside (DDM) to rapidly and easily prepare the digested virus proteins.

### CROSS-REFERENCE TO RELATED APPLICATIONS

This application is a U.S. national stage application of PCT/EP2022/078725 filed 14 Oct. 2022, which claims the benefit of, and relies on the filing date of, U.S. Provisional Patent Application No. 63/256,111, filed 15 Oct. 2021, European Patent Application No. 21205732.7 filed 29 Oct. 2021, and U.S. Provisional Patent Application No. 63/368,803, filed 19 Jul. 2022, the entire disclosures of which are incorporated herein by reference.

### SEQUENCE LISTING

The instant application contains a Sequence Listing which has been submitted electronically in XML format and is hereby incorporated by reference in its entirety. Said XML copy, created on 15 Apr. 2024, is named DFMP-135-PCT-US Sequence Listing.xml and is 66,293 bytes in size.

### FIELD OF THE INVENTION

The present disclosure provides methods for preparing digested virus proteins, including adenovirus and adeno-associated virus capsid proteins, from a sample of virus proteins, as well as methods of analyzing such digested virus proteins via liquid chromatography-tandem mass spectrometry. The methods include the use of a mixture of sodium deoxycholate (SDC) and N-dodecyl-beta-D-Maltoside (DDM) to rapidly and easily prepare the digested virus proteins.

### BACKGROUND OF THE INVENTION

Recombinant adeno associated virus (AAV) vectors have become excellent choices for gene therapy applications, with high safety and efficiency due to low toxicity, availability of viral serotypes and stable gene expression. See e.g., Flotte, T. R., Gene therapy progress and prospects: recombinant adeno-associated virus (rAAV) vectors. Gene Ther 2004, 11 (10), 805-10.

AAVs are composed of single-stranded DNA encased in an icosahedral protein capsid shell. The capsid is composed of 60 subunits of three viral proteins (VP) (VP1, VP2 and VP3) in an approximate molar ratio of 1:1:10 that share a common C-terminal amino acid sequence.

Robust, convenient analytical approaches to determine the heterogeneity of the capsid viral proteins are required to complement recently developed methods for the production of recombinant AAVs from producer cell lines including Sf9 insect and human embryonic kidney HEK293 cells. For example, the US Food and Drug Administration (FDA) recommends the identification of gene therapy products (e.g. AAV serotypes) from other products in the same facility. The most common current techniques for identifying AAV serotypes are enzyme-linked immunosorbent assays (ELISAs) and immunoblotting. However, both techniques lack sufficient sensitivity for products with high degrees of similarity, and highly specific antibodies must be generated for each type of AAV.

Several sample preparation strategies that enable efficient protein extraction and digestion before mass spectrometric analysis in proteomic studies have been developed, involving the use of detergents or chaotropic agents, followed by desalting or dilution, which is required to preserve protease activity of the digesting enzyme. However, the multiple work-up steps inevitably lead to substantial sample loss and generate high volumes of digested VP material at low concentration that cannot be loaded onto the LC column with a single injection for in-depth LC-MS/MS characterization of the trace VPs. Moreover, AAV analysis requires additional sample handling steps (and consequent losses) to those in a standard proteomic study, including denaturation of the capsid with acetic acid to release the VPs followed by exchange of the buffer to one that is compatible with proteomic sample work-up. Protocols including such multi-step sample preparation can provide accurate structural information, but they are time-consuming and lack robustness, and thus are not suitable for routine quantitative analyses.

Protein precipitation has been widely used for isolating proteins from diverse matrices, but its optimization remains a major challenge due to variations in target proteins (chemistry and concentration), matrices (particularly ionic strength of the formulation buffer) and variable parameters, such as optimal incubation time, temperature and type of organic solvent. All of these parameters can affect the performance of the method and might lead to low or inconsistent protein recovery. According to a recent study, optimized conditions for acetone precipitation of proteins from yeast lysates, affording 98+1% recovery, include addition of sodium chloride (1 to 100 mM) and a short incubation time (2 min) at room temperature (RT). However, an optimal procedure for precipitating proteins from defined samples may lead to substantial loss of proteins in other samples with different matrices. Thus, specific optimization is crucial, particularly for VPs, as they are present at very low concentrations in complex matrices.

In-depth characterization of the three capsid viral proteins (VPs 1, 2 and 3) of adeno-associated viruses is urgently needed to ensure the consistency of gene therapy products and processes. These proteins are typically present at very low concentrations in matrices containing high concentrations of excipients and salts. Thus, there is a need for convenient methods for sample preparation before proteomic analysis.

Similarly, Adenoviruses (AdVs) have recently become widely used therapeutic vectors for severe acute respiratory syndrome coronavirus 2 (SARS-COV-2) vaccine. AdVs are large, non-enveloped viruses with an icosahedral capsid formed from several proteins that encloses double-stranded DNA. Alteration of the type of cell line used or scale of production and purification can affect AdVs' composition and influence the interactions of virus particles with cells, and hence the products' biological activity and potency. The VPs are the main components and key players in initial stages of infection by the virus particles, so their heterogeneity and content must be evaluated to ensure product and process consistency. Peptide mapping can provide detailed information on these proteins, e.g., their amino acid sequences and post-translational modifications (PTMs), which is crucial for development and optimization of the manufacturing processes. However, sample preparation remains the main bottleneck for successful proteomic analysis of the viral proteins (VPs) of AdVs due to their low concentrations and vast stoichiometric ranges.

The present invention provides a fast, reproducible VP sample preparation approach, involving protein precipitation followed by re-dissolving in sodium deoxycholate (SDC)/N dodecyl-beta-D-Maltoside (DDM), enabling generation of low-volume trypsin digests without further clean-up steps. The compatibility of this precipitation method was further assessed by dissolving the resulting protein pellet in guanidine hydrochloride (Gu-HCl) followed by Asp-N digestion. 100% and 99.2% sequence coverage of AAV VP1 were obtained using this approach with trypsin and Asp-N digestion, respectively. In addition, N- and C-terminal amino acid sequences of AAV VP1, VP2, and VP3 with their PTMs were completely characterized.

The use of lower SDC/DDM concentrations, as described herein, obviated removal of SDC and enabled identification of all main structural proteins of AdV5 with high amino acid sequence coverage (92% of amino acids in Adv5 VPs on average) and quantification of 53 PTMs in a single LC-MS/MS experiment using trypsin protease.

The presented method is highly reproducible, robust, and suitable for the proteomic study of viruses, such as AAV and AdV serotypes. Furthermore, it is not labor-intense and can easily be adapted for both high and low amounts of starting materials.

### SUMMARY OF THE INVENTION

In some embodiments, provided herein is a method of preparing a digested virus protein, comprising: precipitating a virus protein from a sample containing the virus protein, dissolving the virus protein in a mixture comprising sodium deoxycholate (SDC) and N-dodecyl-beta-D-Maltoside (DDM) to generate a solution, and digesting the virus protein with a protease.

In further embodiments, provided herein is a method of analyzing a digested virus protein, comprising: precipitating a virus protein from a sample containing the virus protein, dissolving the virus protein in a mixture comprising sodium deoxycholate (SDC) and N-dodecyl-beta-D-Maltoside (DDM) to generate a solution, digesting the virus protein with a protease, removing the SDC from the solution, and analyzing the digested virus protein via liquid chromatography-tandem mass spectrometry (LC-MS/MS).

In embodiments, the step of removing the SDC from the solution can be omitted because it has been found that lower concentrations of SDC do not interfere with the LC-MS/MS analysis. Accordingly, also provided herein is a method of analyzing a digested virus protein, comprising: precipitating a virus protein from a sample containing the virus protein; dissolving the virus protein in a mixture comprising sodium deoxycholate (SDC) and N-dodecyl-beta-D-Maltoside (DDM) to generate a solution; digesting the virus protein with a protease; and analyzing the digested virus protein via liquid chromatography-tandem mass spectrometry (LC-MS/MS).

In embodiments, the virus protein is an adeno-associated virus capsid protein (AAV capsid protein), an adenovirus protein, a lentivirus protein, a retrovirus protein, or a herpes simplex virus protein. Suitably, the virus protein is an AAV capsid protein. In additional embodiments, the virus protein is an adenovirus protein, for example, an adenovirus 5, 26, 35 or 48 protein. Suitably, the adenovirus protein is adenovirus 5 protein. In exemplary embodiments, the virus protein is a lentivirus protein.

Suitably, the virus protein is dissolved in a mixture comprising SDC at about 0.01% to 1.5% (w/w) and DDM at about 0.01% to 1.0% (w/w). For example, the mixture comprises SDC at about 0.5% to 1.5% (w/w) and DDM at about 0.01% to 1% (w/w). In embodiments, the mixture comprises SDC at about 0.5% to 1.5% (w/w) and DDM at about 0.2% to 1.0% (w/w) or the mixture comprises SDC at about 0.75% to 1.25% (w/w) and DDM at about 0.5% to 0.8% (w/w). Suitably, the mixture comprises a ratio of about 1:0.5 w/w (SDC: DDM). The mixture may also comprise lower detergent concentrations, such as SDC at about 0.01% to 0.6% (w/w) and DDM at about 0.01% to 1% (w/w). In embodiments, the mixture comprises at about 0.01% to 0.6% (w/w) and DDM at about 0.01% to 0.6% (w/w) or the mixture comprises SDC at about 0.2% to 0.4% (w/w) and DDM at about 0.05% to 0.2% (w/w). Suitably, such mixture may comprise a ratio of about 3.5:1 w/w (SDC: DDM).

In embodiments, the dissolving in a solution occurs at about pH 6.0 to about pH 9.0. Suitably, the digesting takes place at about 30° C. to 40° C., for a period of about 2 to 12 hours. In exemplary embodiments, the precipitating comprises precipitation with chloroform/methanol/water and centrifugation. Suitably, the digesting comprises digesting with trypsin. In embodiments, the digesting is done at a ratio of about 20:1 to about 100:1 w:w of virus protein: trypsin.

Suitably, the digested virus protein is about 3 to 70 amino acids in length.

In exemplary embodiments, the analyzing comprises injecting the digested virus protein into a Liquid Chromatography Mass Spectrometer, without first performing a buffer exchange or a desalting step. Suitably, a solution volume that is analyzed by LC-MS/MS is less than 50 μL. In suitable embodiments, the sample containing the virus protein has a concentration of virus protein of about 0.001 mg/mL to about 0.10 mg/mL.

### BRIEF DESCRIPTION OF THE DRAWINGS

FIG. 1 shows an overview of a virus protein extraction and analysis protocol as described herein.

FIGS. 2A to 2G show total ion current (TIC) chromatograms of a monoclonal antibody A (mAb A) with indicated percentage of SDC/DDM.

FIGS. 3A to 3C show the results of LC-UV-MS analysis of Anc80 capsid viral proteins.

FIG. 4 shows extracted ion chromatogram (upper panel) and MS/MS spectra (bottom panel) of the tryptic peptide of T23

FIG. 5 shows extracted ion current (XIC) and MS/MS spectra of the tryptic peptide T33.

FIGS. 6A to 6C show MS/MS spectra of the N- and C-terminal peptides of VP1.

FIGS. 7A to 7D show MS/MS spectra of N-terminal amino acids of VP2 and associated PTMs after Asp-N digestion.

FIG. 8 shows MS/MS spectra of the N-terminal amino acid sequence of VP3.

FIG. 9 shows an overview of a modified virus protein extraction and analysis protocol, which does not require removal of SDC, as described herein.

FIG. 10 shows total ion current (TIC) chromatograms of monoclonal antibody A (mAb A). TICs of (A) the supernatant after SDC removal and (B) washing of the SDC pellet with water.

FIG. 11 shows TIC chromatograms of trypsin-digested mAb A following processing with 0.1%-0.4% w/v SDC and 0.1% w/v DDM without SDC removal (top seven panels) or with 1% w/v SDC and 0.5% w/v DDM and additional SDC removal.

FIG. 12 shows extracted ion currents (XICs) of eight selected peptides of monoclonal antibody A (mAb A) following processing with 0.1%-0.4% w/v SDC and 0.1% w/v DDM without SDC removal (top seven panels) or with 1% w/v SDC and 0.5% w/v DDM and additional SDC removal.

FIG. 13 shows MS/MS spectra of N-terminal amino acid sequences of pIX and pVI. (A) Acetylated peptides of the N-terminus of pIX without methionine and with acetylation of the serine. (B) Acetylated methionine and (C) acetylated and oxidized methionine from the N-terminal peptides of pVI. Enlarged mass spectra of the b1 ions are displayed in B and C, with the observed mass shift (+16 Da) in the corresponding insets.

FIG. 14 shows typical MS/MS spectra of (A) phosphorylated and (B) deamidated peptides of VPs of AdV5.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation, for example depending on the degree of accuracy of a measuring method.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps.

Liquid chromatography-tandem mass spectrometry (LC-MS/MS) is a powerful technique to study VPs' structures and their post-translational modifications (PTMs). However, preparation of VP samples for characterization by LC-MS/MS is challenging, as these proteins are typically present at very low concentration in a matrix that often contains excipients and high concentration of salts. Thus, efficient sample preparation is crucial for precise and accurate results.

The methods described herein include a sample preparation approach, involving protein precipitation followed by re-dissolving in a mixture of sodium deoxycholate (SDC) and N-dodecyl-beta-D-Maltoside (DDM) enabling generation of low-volume digests without further clean-up steps for virus proteins analysis via liquid chromatography-tandem mass spectrometry.

In exemplary embodiments, provided herein is a method of preparing a digested virus protein (VP), comprising:
a) precipitating a virus protein from a sample containing the virus protein;
b) dissolving the virus protein in a mixture comprising sodium deoxycholate (SDC) and N-dodecyl-beta-D-Maltoside (DDM) to generate a solution; and
c) digesting the virus protein with a protease.

Suitably, steps a) to c) of the method described herein, are done consecutively in this order, a) to c). As described herein, the result of step a) provides a precipitate containing precipitated virus protein. The precipitate of step a) is dissolved in step b). In step c), the virus protein present in the solution of step b) is digested with a protease, providing a solution of digested virus protein, that is a solution of peptides.

Also provided herein is a method of analyzing a digested virus protein, comprising:
a) precipitating a virus protein from a sample containing the virus protein;
b) dissolving the virus protein in a mixture comprising sodium deoxycholate (SDC) and N-dodecyl-beta-D-Maltoside (DDM) to generate a solution;
c) digesting the virus protein with a protease;
d) removing the SDC from the solution; and
e) analyzing the digested virus protein via liquid chromatography-tandem mass spectrometry (LC-MS/MS).

Suitably, steps a) to e) of the analysis method described herein, are done consecutively in this order, a) to e). As described herein, the result of step a) provides a precipitate containing precipitated virus protein. The precipitate of step a) is dissolved in step b). In step c), the virus protein present in the solution of step b) is digested with a protease, providing a solution of digested virus protein, that is a solution of peptides. In step d) the SDC is removed from the solution of digested virus protein providing a solution ready for analysis. In step e) the digested virus protein is analyzed.

Step d) can be omitted if the SDC concentration is sufficiently low so as not to interfere with LC-MS/MS analysis. Accordingly, also provided herein is a method of analyzing a digested virus protein, comprising:
a) precipitating a virus protein from a sample containing the virus protein;
b) dissolving the virus protein in a mixture comprising sodium deoxycholate (SDC) and N-dodecyl-beta-D-Maltoside (DDM) to generate a solution;
c) digesting the virus protein with a protease; and
e) analyzing the digested virus protein via liquid chromatography-tandem mass spectrometry (LC-MS/MS).

Suitably, steps a) to e) of the analysis method may be done consecutively in this order, a) to c) and e). As described herein, the result of step a) provides a precipitate containing precipitated virus protein. The precipitate of step a) is dissolved in step b). In step c), the virus protein present in the solution of step b) is digested with a protease, providing a solution of digested virus protein, that is a solution of peptides. In step e) the digested virus protein is analyzed.

As used herein, the term “virus protein” refers to a protein that forms part of a virus, for example a protein that forms a structural component of a virus, such as a capsid protein of a virus.

Example of virus proteins or viral proteins, that can be prepared and analyzed according to the methods described herein include, but are not limited to, an adeno-associated virus capsid protein (AAV capsid protein), an adenovirus protein, a lentivirus protein, a retrovirus protein, and a herpes simplex virus protein.

In suitable embodiments, the virus protein is an adeno-associated virus (AAV) protein, in particular an AAV capsid protein. AAVs are composed of single-stranded DNA encased in an icosahedral protein capsid shell. The capsid is composed of 60 subunits of three viral proteins (VP1, VP2 and VP3) in an approximate molar ratio of 1:1:10 that share a common C-terminal amino acid sequence.

In further embodiments, the virus protein is an adenovirus protein Adenovirus contain double-stranded DNA inside an icosahedral capsid with a total molecular weight of about 150 MDa. Human Adenovirus capsid is composed of 13 different proteins referred to herein as virus proteins “VPs”, which are categorized as major proteins (Hexon, Penton base and Fiber), cement/minor proteins (pIIIa, pVI, pVIII and pIX) and core proteins (pV, pVII, pTP, pu, AVP and pIVa2). Major and minor proteins account for the highest and lowest percentages of the total weight of the AdV5 proteins (˜64.1% and ˜15.6%, respectively). In exemplary embodiments, the adenovirus protein is an adenovirus 5, 26, 35 or 48 protein.

Lentivirus contains a single stranded RNA genome with a reverse transcriptase enzyme. Exemplary lentivirus proteins are known in the art.

It should be understood that “an AAV capsid protein” is meant to include more than one AAV capsid protein, including different ratios and amounts of the three viral proteins from AAV. Similarly, an “AdV VP” is meant to include more than one AdV protein, including different ratios and amounts of the 13 viral proteins from AdV.

As used herein “precipitating” refers to a method in which proteins from a virus, including AAV capsid proteins, are removed from a solution to allow further analysis and characterization, including via various instrumentation such as mass spectrometry, etc. In embodiments, the methods suitably comprise precipitating a virus protein from a sample containing the virus protein. A “sample” refers to the product of any bioreaction that produces a virus, including adenovirus, lentivirus, retrovirus, herpes simplex virus, or AAV, and suitably refers to the production of viruses from one or more cell lines, including for example, human embryonic kidney (HEK) cells, including HEK-293, Sf9 cell line, HeLa cells, etc.

Various methods of precipitating virus proteins, are known in the art. For example, virus proteins can be precipitated using a chloroform/methanol/water precipitation technique, including centrifugation to form a protein pellet. Such methods include the sequential addition of methanol, chloroform and water to a sample, with short vortex-mixing and fast centrifugation steps (10 sec at 14′000 g) following each addition. A protein precipitate generally appears at the interface as a white layer between upper and lower phases. The upper phase can be removed and discarded, then cold methanol is added to the remaining mixture. After centrifugation the supernatant is removed. Finally, the pellet is dried by vacuum centrifugation.

Additional precipitation methods include the use of cold acetone, followed by storage in a freezer (e.g., about 60 minutes), centrifugation, removal of the supernatant, and then drying of the protein pellet.

Following the precipitation of the virus protein, the virus protein is dissolved in a mixture comprising sodium deoxycholate (SDC) and N-dodecyl-beta-D-Maltoside (DDM) to generate a solution. As used herein, “dissolving” the virus protein means the generation of a working solution of the virus proteins in the mixture comprising SDC and DDM, but does not necessary require complete dissolution of the virus proteins. Instead, dissolving can also include the generation of a suspension, or near suspension, of the virus proteins in the mixture (i.e., while a clear solution may form, a cloudy or slightly precipitated solution/suspension can also occur upon the use of the SDC/DDM mixture).

As described herein, it has been surprisingly found that the use of a mixture of SDC and DDM as a part of a virus protein digestion, significantly reduces sample processing steps and makes it accessible for a broad range of biological laboratories, with no need for high expertise in proteomic workflows. In exemplary embodiments, the virus protein is dissolved in a mixture comprising SDC at about 0.01% to 1.5% (w/w) and DDM at about 0.01% to 1.0% (w/w).

In embodiments, the virus protein is dissolved in a mixture comprising SDC at about 0.5% to 1.5% (w/w) and DDM at about 0.01% to 1.0% (w/w), preferably comprising SDC at about 0.5% to 1.5% (w/w) and DDM at about 0.2% to 1.0% (w/w), more preferably comprising SDC at about 0.5% to 1.5% (w/w) and DDM at about 0.4% to 1.0% (w/w). The mixture is an aqueous mixture. For all percentages expressed as w/w, the percentages refer to the weight of the component, relative to the total weight of the mixture. For example, the amount of SDC in the mixture can be about 0.4% to 1.6%, or about 0.5% to 1.5%, or about 0.6% to 1.5%, about 0.75% to 1.25%, about 0.8% to 1.2%, about 0.9% to 1.1%, or about 0.7%, about 0.8%, about 0.9%, about 1.0%, about 1.1% or about 1.2% (w/w). The amount of DDM in the mixture can be about 0.01% to 1.0% (w/w), or about 0.02% to 1.0% (w/w), or about 0.05% to 1.0% (w/w), or about 0.1% to 0.6%, or about 0.2% to 1.0% (w/w), or about 0.3% to 1.1% (w/w), or about 0.4% to 1.0%, about 0.5% to 0.9%, about 0.5% to 0.8%, about 0.5% to 0.7%, about 0.5% to 0.6%, or about 0.4%, about 0.5%, about 0.6%, about 0.7%, about 0.8% or about 0.9% (w/w).

In suitable embodiments, the mixture comprises SDC at about 0.75% to 1.25% (w/w) and DDM at about 0.5% to 0.8% (w/w), and more suitably, the mixture comprises a ratio of about 1:0.5 (w/w) (SDC: DDM).

The mixture of SDC and DDM is prepared in a suitable buffer, including for example a bicarbonate buffer, such as ammonium bicarbonate, e.g., about 30 mM to 100 mM, or about 30 mM to 70 mM, preferably about 50 mM ammonium bicarbonate, having a pH of 6 to 9, or about pH 8. Additional buffers known in the art can also be used to prepare the mixture, such as Tris-HCl buffer. Suitably, the mixture is prepared at a pH of about pH 6.0 to about pH 9.0, such that the dissolving of the virus proteins occurs at a pH of about pH 6.0 to about pH 9.0, suitably a pH of about pH 7 to about pH 9, more suitably at about pH 8.

The method further includes digesting the virus protein with a protease. As used herein a “protease” refers to an enzyme that breaks down a protein, and specifically an enzyme capable of breaking down a virus protein. Exemplary proteases for use in the methods described here including, for example trypsin, Asp-N, Lys-C, Lys-N, chymotrypsin, or Glu-C protease, preferably trypsin or Asp-N. Additional proteases that can be used in the methods described herein are known in the art.

Digestion times and conditions with the protease will vary based on the protease selected. However, if trypsin is utilized, the digestion generally takes place at about 30° C. to 40° C. (suitably about 37° C.) for a period of about 2 to 12 hours, including about 2 to about 4 hours, e.g., about 3 hours. The amount of protease used will also vary based on the selected enzyme. For trypsin digestion, the protease is suitably used at a ratio of about 20:1 to about 100:1 w:w, or of about 20:1 to about 40:1 w:w, more suitably about 30:1 w:w, where the ratio is a weight ratio of virus protein to trypsin (virus protein:trypsin). Suitably, the proteins are dissolving at about pH 6.0 to about pH 9.0, and at about 30° C. to 40° C. Dissolving is usually a fast step in the range of a few minutes, but can extend up to a period of about 2 to 12 hours, for example for about 2 to 4 hours, e.g., about 3 hours.

The protease digestion may be stopped using suitable methods, including for example the addition of acids, e.g. Trifluoroacetic acid (TFA) or Difluoroacetic acid (DFA) in the case of trypsin, and formic acid for Asp-N.

In embodiments, following the digestion of the virus protein with a protease, SDC is suitably removed from the solution. This solution can then be utilized in an analysis protocol, including via LC-MS/MS, as described herein, or stored if desired for later analysis. SDC can be removed from digests, e.g., by means of acidification (i.e. acid precipitation). Methods for removing SDC from the solution suitably include separating the SDC (which may appear as a slight cloudiness) via centrifugation, and removing the supernatant which contains the virus protein. However, this step also leads to a loss of peptides due to issues of handling and because peptides, in particular long hydrophobic ones, may co-precipitate with the SDC. For example, experiments described herein have shown that the washing liquor of the SDC precipitate still contains about 20% of the peptides previously present in the mixture. This is particularly undesirable when analyzing proteins present in low amounts, as may occur in the analysis of gene therapy vectors, e.g. AdVs.

As further described herein, it has been surprisingly found that the use of a mixture of SDC and DDM at lower concentrations obviates the need for SDC removal (which may otherwise suppresses electrospray ionization and interfere with chromatographic separation of the peptides), thus reducing sample loss and eliminating another time-consuming processing step, while still allowing for excellent protein solubilization as well as denaturation for subsequent protease (e.g., trypsin) cleavage. As an additional advantage, such mixture can be directly analyzed using LC-MS/MS after the digestion step without stopping the protease digestion reaction. Thus, in other exemplary embodiments, the virus protein is dissolved in a mixture comprising SDC at about 0.01% to 0.6% (w/w) and DDM at about 0.005% to 1.0% (w/w), preferably comprising SDC at about 0.01% to 0.6% (w/w) and DDM at about 0.005% to 1.0% (w/w), more preferably comprising SDC at about 0.01% to 0.5% (w/w) and DDM at about 0.01% to 1.0% (w/w). More specifically, the mixture may comprise SDC at about 0.01% to 0.5% (w/w) and DDM at about 0.01% to 0.6% (w/w). In such embodiments, the method may suitably not comprise a step d) of removing the SDC from the solution. The mixture is an aqueous mixture. For all percentages expressed as w/w, the percentages refer to the weight of the component, relative to the total weight of the mixture. For example, the amount of SDC in the mixture can be about 0.01% to 0.6%, or about 0.02% to 0.5%, or about 0.05% to 0.5%, about 0.1% to 0.5%, about 0.2% to 0.4%, about 0.3% to 0.4%, or about 0.15%, about 0.2%, about 0.25%, about 0.3%, about 0.35% or about 0.4% (w/w). The amount of DDM in the mixture can be about 0.005% to 1.0% (w/w), about 0.01% to 1.0% (w/w), or about 0.01% to 0.8% (w/w), or about 0.01% to 0.6%, about 0.02% to 0.6%, about 0.05% to 0.6%, about 0.05% to 0.5%, about 0.05% to 0.2%, or about 0.05%, about 0.1%, about 0.2%, about 0.3%, about 0.4% or about 0.5% (w/w).

In suitable embodiments, the mixture comprises SDC at about 0.2% to 0.4% (w/w) and DDM at about 0.05% to 0.2% (w/w). Suitably, the mixture comprises a ratio of about 3.5:1 (w/w) (SDC: DDM).

The modified method using lower concentrations of SDC and DDM thus represents a significant improvement in terms of the required sample processing steps and reduced sample loss, while preserving sequence coverage due to the ability of the SDC/DDM mixture to increase potential protease (e.g., trypsin) cleavage sites and enhance the solubility of hydrophobic peptides.

As described herein, it has been surprisingly found that the use of a mixture of SDC and DDM as a part of a virus protein digestion process does not require any clean up steps following the digestion.

Removal of detergents significantly improves the analysis of the virus proteins via LC-MS/MS, as it is well known that detergents can significantly interfere with reversed-phase (RP) chromatography and MS analysis of virus proteins. Examples of detergents that can be removed via the methods described herein include various poloxamers (non-ionic triblock copolymers), as well as other non-ionic detergents such as Triton X-100, NP-40, Tween 20 and Tween 80.

The methods described herein allow for the preparation, and ultimate analysis, of digested virus proteins having amino acid lengths of up to 100 amino acids. For example, the virus proteins that are extracted, digested with different protease and ultimately analyzed, suitably include peptides having 3 to 100 amino acids in length, including 3 to 90 amino acids in length, 3 to 80 amino acids in length, 3 to 70 amino acids in length, 3 to 60 amino acids in length, 3 to 50 amino acids in length, or 3 to 40 amino acids in length.

In embodiments, the methods described herein further comprise analyzing the virus proteins via liquid chromatography-tandem mass spectrometry (LC-MS/MS), following the digesting of the virus protein with a protease. In such embodiments, the methods can further include removing the SDC from the solution, following the digesting, but prior to the analyzing. The removal can be done by centrifugation with subsequent separation of the protein containing fraction for subsequent analysis by LS-MS/MS.

In additional embodiments, provided herein is a method of analyzing virus protein. As described herein, methods of analyzing virus proteins allow for the complete sequence confirmation of the proteins, as well as characterization of the amino acid sequences of the N- and C-terminal regions of the virus proteins, along with information regarding their post-translational modifications. In embodiments where the analyzed virus proteins are virus capsid proteins (including AAV capsid proteins), the analysis methods described herein provide high-confidence confirmation of capsid identity, and can distinguish serotypes with lower than 10 Da mass differences in the capsid proteins.

The methods of analyzing suitably include precipitating virus protein from sample including the virus protein, dissolving the virus protein in a mixture comprising sodium deoxycholate (SDC) and N-dodecyl-beta-D-Maltoside (DDM) to generate a solution, digesting the virus protein with a protease, suitably removing the SDC from the solution, if required, and analyzing the virus protein via liquid chromatography-tandem mass spectrometry (LC-MS/MS).

As described herein, the mixture for use in dissolving the virus protein suitably comprises SDC at about 0.01% to 1.5% (w/w) and DDM at about 0.01% to 1.0% (w/w). In exemplary embodiments, the mixture comprises SDC at about 0.5% to 1.5% (w/w) and DDM at about 0.2% to 1.0% (w/w) or the mixture comprises SDC at about 0.75% to 1.25% (w/w) and DDM at about 0.5% to 0.8% (w/w), and suitably the mixture comprises a ratio of about 1:0.75 w/w (SDC: DDM). The mixture may also comprise lower detergent concentrations, such as SDC at about 0.01% to 0.6% (w/w) and DDM at about 0.01% to 1.0% (w/w). Such mixture may also comprise SDC at about 0.01% to 0.6% (w/w) and DDM at about 0.01% to 0.6% (w/w), or the mixture comprises SDC at about 0.2% to 0.4% (w/w) and DDM at about 0.05% to 0.2% (w/w). Suitably the mixture may comprise a ratio of about 3.5:1 w/w (SDC: DDM). In embodiments using lower detergent concentrations, the method may suitably not comprise a step d) of removing the SDC from the solution.

Various methods for precipitating virus proteins are described herein, including the use of precipitation with chloroform/methanol/water and centrifugation. Suitably, the virus proteins are digested with a protease, including for example trypsin. Exemplary methods for trypsin digestion are described herein, and include digesting with trypsin at a ratio of about 20:1 to about 100:1 w:w (virus protein: trypsin).

As described herein, liquid chromatography-tandem mass spectrometry (LC-MS/MS) is a powerful technique to study the structures and PTMs of virus proteins. However, preparation of virus protein samples (including virus capsid proteins) for characterization by LC-MS/MS is challenging, as these proteins are typically present at very low concentration in a matrix that often contains excipients and high concentration of salts. For example, nonionic detergents, such as poloxamer, are frequently used to improve the manufacturing process in order to increase the yield in gene therapies. Even when present at very low concentration (e.g., 0.001%, w/v), poloxamer can strongly interfere with reversed-phase (RP) chromatography and MS analysis of VPs. Thus, efficient sample preparation is crucial for precise and accurate results, and as described herein, the methods substantially remove any detergent present in the virus sample, including poloxamers.

Methods for analyzing virus proteins are described throughout. Suitably, the analysis methods include injecting the digested virus protein into a Liquid Chromatography Mass Spectrometer, but first with performing a buffer exchange or a desalting step. As described herein, the use of a mixture of SDC and DDM allows for the omission of such buffer exchange and desalting steps, significantly decreasing the time and expense of virus protein analysis methods, and reducing the complexity of such methods. Furthermore, as pointed out above, it has been found by the inventors that lower concentrations of an SDC/DDM mixture as described herein are entirely compatible with LC-MS/MS analysis and may therefore remain in the sample.

The analysis methods described herein suitably utilize a solution volume for analysis via LC-MS/MS is less than 50 μL. In embodiments, the solution volume that is used for analysis via LC-MS/MS is about 3 μL to about 50 μL, or about 10 μL to about 50 μL, or about 20 μL to about 50 μL.

The methods of extraction and analysis can be used with sample having a concentration of virus protein of about 0.001 mg/mL to about 0.10 mg/mL. As described throughout, the methods are suitably used for analysis of digested virus proteins having a length of about 3 to 70 amino acids.

### EXAMPLES

### Example 1: Extraction and Analysis of AAV Capsid Proteins

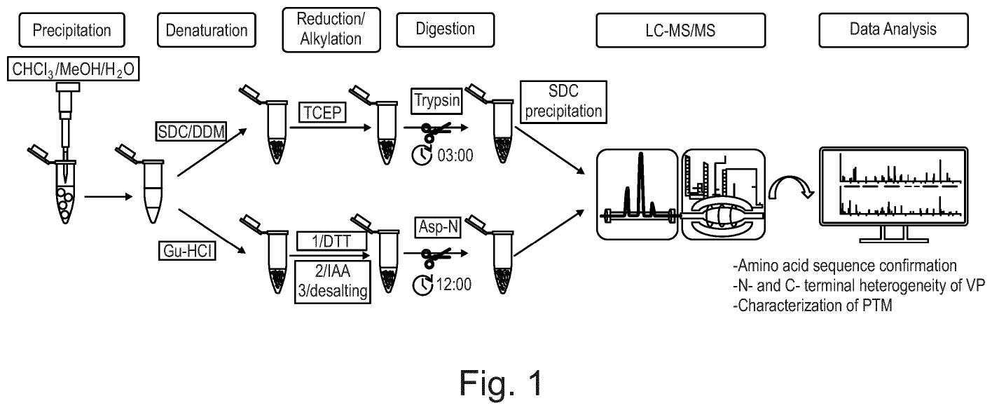

FIG. 1 shows an overview of the extraction and analysis protocols outlined in the following Example. The workflow includes concentration of Anc80 AAV samples with a cut-off filter to reduce their volume. Capsid proteins are precipitated, the protein pellets are dissolved in the selected denaturation reagent, and the proteins are digested using either trypsin or Asp-N. Finally, generated peptides are analyzed by LC-MS/MS. The SDC/DDM proportion was optimized for trypsin digestion, and compared two common precipitation approaches.

### Materials and Methods

### Chemicals

Dithiothreitol (DTT), tris 2-carboxyethyl phosphine (TCEP), ammonium bicarbonate (ABC), iodoacetamide (IAA), ultrapure formic acid, acetic acid, guanidine-HCl (Gu-HCl), Tris-HCl, acetone, methanol, acetonitrile (ACN), water, trifluoroacetic acid (TFA), sodium deoxycholate (SDC) and Tris base were purchased from Sigma-Aldrich (St. Louis, MO). Amicon ultra-4 filters (10 kDa MWCO) were purchased from Millipore (Billerica, MA). Sequencing grade trypsin was purchased from Promega (Milwaukee, WI). Asp-N protease was purchased from Roche Diagnostics (Indianapolis, IN). Zeba spin desalting columns (7K MWCO, 0.5 mL) and N-dodecyl-beta-D-Maltoside (DDM) were purchased from Thermo Fisher Scientific (Waltham, MA), and difluoroacetic acid (DFA) from Waters (Milford, MA).

### Vector Production and Purification

Anc80 samples were produced and purified on a scalable manufacturing platform at Lonza Houston, Inc. A transient triple transfection of suspension HEK293 cells was used, as described in Bingnan Gu et al., Cell & Gene Therapy Insights 2018, 4 (S1), 753-769, DOI: 10.18609/cgti.2018.080, to produce Anc80 in a 250 L single use bioreactor and an aliquot of the recombinant Anc80 was purified using affinity chromatography followed by ion exchange chromatography providing samples of purified Anc80.

### Sample Preparation

Samples of the Anc80 AAV capsid proteins were prepared as described below. A recombinant human monoclonal IgG4 antibody (designated mAb A) was also produced and purified at Lonza using standard manufacturing procedures.

100 μL samples of purified Anc80 prepared as described above containing ca. 1 μg of the Anc80 AAV capsid proteins (VPs) were subjected to intact protein analysis as described below after reducing their volume from 100 μL to 20 μL, denaturing them by adding 2 μL of acetic acid and incubating the resulting mixtures at RT for 10 min prior to LC-MS analysis for the intact protein analysis as described below.

Peptide mapping was performed after reducing the volume of 500 μL samples of purified Anc80 prepared as described above (containing roughly 5 μg of Anc80 AAV capsid proteins (VPs)) to 100 μL before protein precipitation as described below providing a so called “100 μL of concentrated sample”. The volumes were reduced by centrifuging samples over 10 kDa cut-off filters, also known as membrane filters, at 10′000 g and 20° C.

### Protein Precipitation by Chloroform/Methanol/Water (Approach 1):

VPs were precipitated with chloroform/methanol/water. Briefly, 400 μL of methanol, 100 μL of chloroform and 300 μL of water were sequentially added to 100 μL of concentrated sample (prepared as described above) with short vortex-mixing and fast centrifugation steps (10 sec at 14′000 g) following each addition. The protein precipitate appeared at the interface as a white layer between upper and lower phases. The upper phase was carefully removed and discarded, then 300 L of cold methanol was added to the remaining mixture. After centrifugation the supernatant was removed. Finally, the protein pellet was dried by vacuum centrifugation.

In all precipitation steps, cold solvents were used, and centrifugation was performed at 14′000 g and 4° C. for 10 min.

### Protein Precipitation by Cold Acetone (Approach 2):

Ice-cold acetone (400 μL) was added to 100 μL concentrated sample (prepared as described above), and the solution was stored for 60 min in a −20° C. freezer. The sample was then centrifuged at 14′000 g and 4° C. for 10 min, the supernatant was removed, and the protein pellet was dried by vacuum centrifugation.

Dissolving with SDC and DDM and Enzymatic Digestion of Capsid Proteins

For trypsin digestion, protein pellets prepared as described above were dissolved in 35 μL of a mixture of SDC (1% w/w) and DDM (0.5% w/w) in 50 mM ammonium bicarbonate (pH 8.0), then vortex-mixed at RT. Proteins were reduced by adding 1 μL of 500 mM TCEP and incubating for 30 min at 50° C., then subjected to trypsin digestion (with a 30:1 w/w protein to protease ratio) for 3 hours at 37° C. The digestion was stopped by adding 1 μL of TFA, followed by cautious mixing. SDC appearing as slight cloudiness was separated by centrifugation (as above), and the supernatant was transferred to an HPLC vial for LC-MS/MS analysis as described below, thereby the SDC was removed.

For Asp-N digestion, protein pellets prepared as described above were dissolved in 6 M Gu-HCl/0.1 M Tris then subjected to reduction and alkylation with DTT and IAA, respectively. Samples were then desalted using Zeba spin filters according to the manufacturer's recommendations, and subjected to protein digestion with Asp-N(at a 30:1 w/w protein to protease ratio) for 12 hours at 37° C. Enzyme activity was stopped by adding 5 μL of formic acid, then the generated peptides were analyzed by LC-MS/MS as described below.

### Liquid Chromatography-Mass Spectrometry

### Peptide-Mapping Analysis

A system consisting of a Vanquish UPLC coupled to an Orbitrap Fusion Lumos mass spectrometer (Thermo Fisher Scientific, Bremen, Germany) was used for all analyses. For peptide-mapping analysis an Acquity UPLC Peptide BEH C18 column (300 Å, 1.7 μm, 2.1 mm×150 mm, Waters Corporation) was used to separate peptides in the digested samples, with a mobile phase consisting of 0.1% v/v formic acid in water (A) and acetonitrile (B). The peptides were eluted with a linear gradient from 1% v/v B to 60% v/v B over 80 min at a flow rate of 0.25 mL/min. The column was then washed with 98% v/v B for 10 min and conditioned with 1% v/v B for 8 min before the next injection.

The mass spectrometer was operated data-dependently in the 200 to 2000 m/z range. Full-scan spectra were recorded with a resolution of 120′000 using an automatic gain control (AGC) target value of 2.0e5 with a maximum injection time of 100 ms. Up to 20 of the most intense ions with 2 to 8 charge states were selected for higher-energy c-trap dissociation (HCD) with a normalized collision energy of 35%. Fragment spectra were recorded at an isolation width of 2.5 Da and a resolution of 15′000 using an AGC target value of 5.0e4 and maximum injection time of 200 ms. Peaks were dynamically excluded from precursor selection for 5 s within a 10 ppm window. Selected peptides were subjected to electrospray ionization with a spray voltage of 3.5 kV and heated capillary temperature of 320° C.

### Intact Protein Analysis

A MAbPac RP column (4 μm, 3.0 mm×100 mm, Thermo Fisher Scientific) operated at 60° C. was used to separate intact VPs, with a mobile phase consisting of 0.1% v/v DFA in water (A) and acetonitrile (B) at a flow rate of 0.25 mL/min. VPs were eluted with a linear gradient of 10% v/v B to 38% v/v B over 54 min following 2 min at 100% v/v B. The column was conditioned at the end of the gradient for 14 min with 10% v/v B before starting the next injection. UV signals from the eluate were recorded at 280 nm.

The mass spectrometer was operated with the following settings: capillary voltage 3.5 kV, surface-induced dissociation (SID) voltage 50%, resolution 17′500 and capillary temperature 320° C. Mass spectra were acquired in positive mode in the m/z 800 to 3′500 range.

### Data Analysis

Raw mass spectral data were processed with Protein Metrics (San Carlos, CA). For peptide mapping, database searching against Anc80 viral capsid protein sequences (VP1, VP2 and VP3) was performed with tolerances of 6 and 20 ppm for peptides detected in MS and MS/MS analyses, respectively. N-terminal acetylation, methionine oxidation, phosphorylation, and asparagine deamidation were included as variable modifications in the searches.

Intact mass spectra were deconvoluted with the following settings: mass range 55′000 to 85′000 Da, minimum difference between mass peaks 15 Da, maximum number of mass peaks 10, and peak sharing disabled.

### Results and Discussion

### Optimization of the SDC/DDM Proportion for Trypsin Digestion

Trypsin is one of the main proteases currently used in bottom-up proteomic studies. However, regardless of the protease used it is important to ensure that target proteins are denatured, the digestion buffer preserves the protease's activity sufficiently, and the matrix containing the digest is compatible with LC-MS/MS analysis. This often demands for multiple buffer exchange or desalting steps. The approach described herein shortens the sample preparation process with the use of SDC, as it can effectively denature proteins without impairing protein digestion, prior to LC-MS/MS analysis. It should be noted that trypsin is active in solutions with up to 10% SDC, but increasing its concentration increases loss of hydrophobic peptides through co-precipitation with the degraded detergent. Such loss is particularly undesirable in trypsin digestion of VPs, which can generate several long peptides that may co-precipitate during SDC precipitation, or be poorly eluted from a desalting column, causing loss of information. In efforts to minimize these potential problems, DDM is used as a combinatorial detergent to increase the solubility of hydrophobic peptides and avoid their co-precipitation with SDC. DDM is a nonionic detergent that is compatible with trypsin digestion and chromatographic separation of the peptides. A high concentration (≥80%) of ACN is required to elute it from a reversed-phase chromatography column, and it does not alter the column's performance during peptide separation. In addition, the peak intensity of DDM eluting at the end of a reversed-phase gradient is low due to its weak ionization in electrospray ionization.

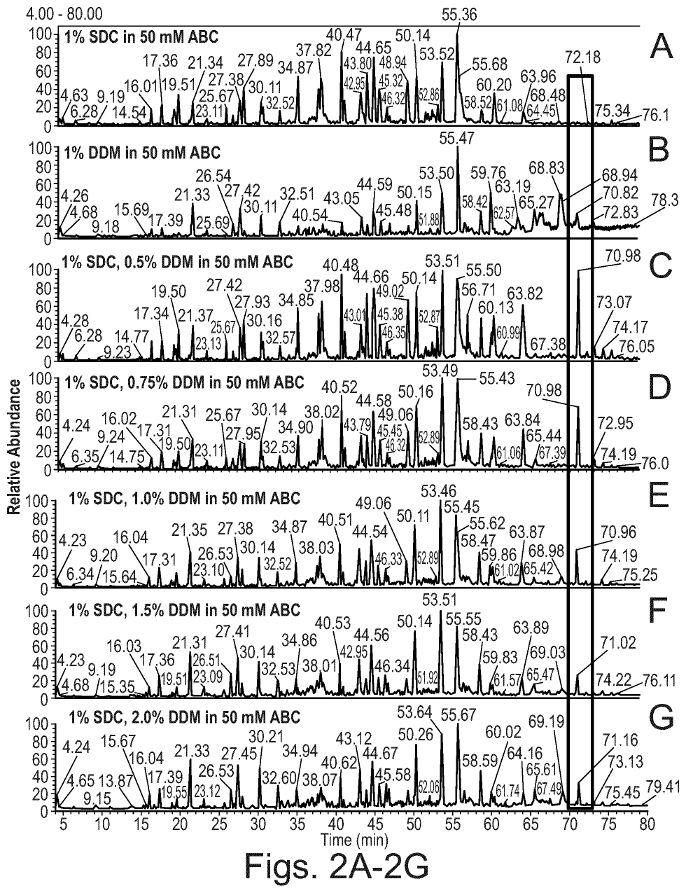

Different concentrations of DDM were first investigated to maximize the recovery of hydrophobic peptides. A monoclonal antibody (mAb A) was denatured using 1% SDC (w/w) with different concentrations of DDM (0.0, 0.5, 0.75, 1.0, 1.5, and 2.0% w/w) then subjected to trypsin digestion, as described in Materials and Methods. A tryptic peptide with 49 amino acids was chosen to evaluate recovery. The peak intensity first increased with increasing DDM concentration, and was maximal when the DDM concentration was at 0.5 to 0.75% (see FIG. 2A to 2G). The similarity of peaks in total ion current (TIC) chromatograms of trypsin digests of mAb A showed that addition of DDM to SDC did not alter trypsin's digestion performance. The TIC of a trypsin digest of mAb A with only DDM revealed incomplete digestion related to DDM's inability to denature proteins. However, use of a higher proportion of DDM is not recommended as it can decrease reversed-phase columns' loading capacities. Therefore, 1% SDC with 0.5% DDM was utilized in the following experiments.

### Performance of Tested Protein Precipitation Approaches

Protein precipitation has two major advantages for VP sample preparations over other commonly used approaches. First, the capsid of AAVs (such as Anc80) may be denatured already directly after addition of organic solvent which is added for the purpose of precipitation, and concomitantly or subsequently to this addition of organic solvent VPs are being precipitated. Therefore, it enables omission of the capsid acid denaturation step and subsequent buffer exchange to a neutral buffer applied in the general proteomics workflow. Second, it can separate the VPs from a wide range of detergents, and the precipitated VPs can be easily dissolved in a low volume of denaturation reagent. Thus, the workflow applying protein precipitation enables analysis of all digested material in a single LC-MS/MS run and in-depth characterization of the VPs, that are typically available in process development or downstream processing samples at low concentrations and/or quantities.

In addition to matrix complexity, differences in amounts of VP1 and VP2 relative to VP3 (due to their approximate 1:1:10, VP1: VP2: VP3 molar ratio) pose major challenges for proteomic studies. Inadequate sample preparation can lead to sample loss, reducing signal-to-noise thresholds for VPs (especially VP1) and hence loss of information. The performance of two frequently applied protein precipitation approaches was compared: precipitation by chloroform/methanol/water (Approach 1) and by acetone (Approach 2). Both approaches require larger organic solvent volumes than the sample volume and are not suitable for samples with volumes larger than 300 μl. Therefore, samples require concentration by a filter membrane with an appropriate molecular weight cut-off before analysis, as described in Material and Methods.

Although protein pellets obtained using both precipitation approaches applied in this study were digested under the same conditions, the results of subsequent LC-MS/MS runs substantially differed. Approach 1 afforded stronger signal intensities of detected peptides and much richer information about the amino acid sequences than Approach 2, with >98% coverage. Another difference between the two approaches was in the solubility of the protein pellet in the SDC/DDM solution. Although the protein pellets obtained using both approaches were dissolved in the same amount of SDC/DDM, the solution obtained with Approach 2 became slightly turbid, whereas pellets obtained with Approach 1 completely disappeared. It has been shown that two-phase precipitation can remove DNA more efficiently than acetone precipitation prior to proteomics analysis, and we hypothesize that the insolubility of pellets obtained using Approach 2 could be related to co-precipitation of DNA with the VPs. Removal of unwanted cellular material such as lipids and DNA is important, as it can substantially interfere with enzymatic digestion and chromatographic separation.

Our results show that precipitation applying Approach 2 does not perform well for VPs in the sample matrix used in this study, whereas Approach 1 yields good results. Therefore, only data obtained using Approach 1 are presented in the following peptide mapping section.

### Intact Protein Analysis

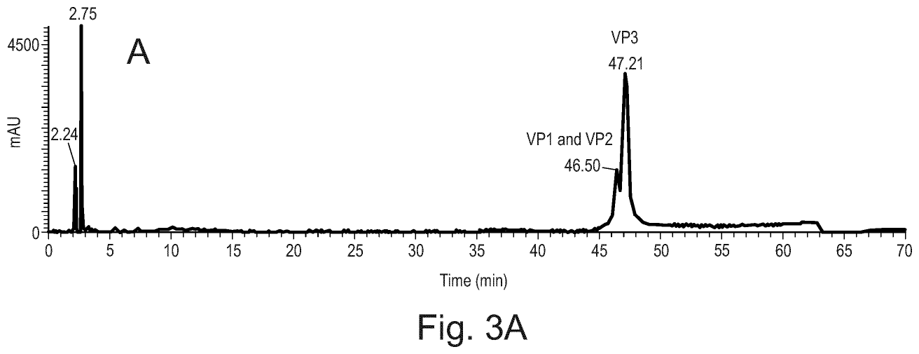

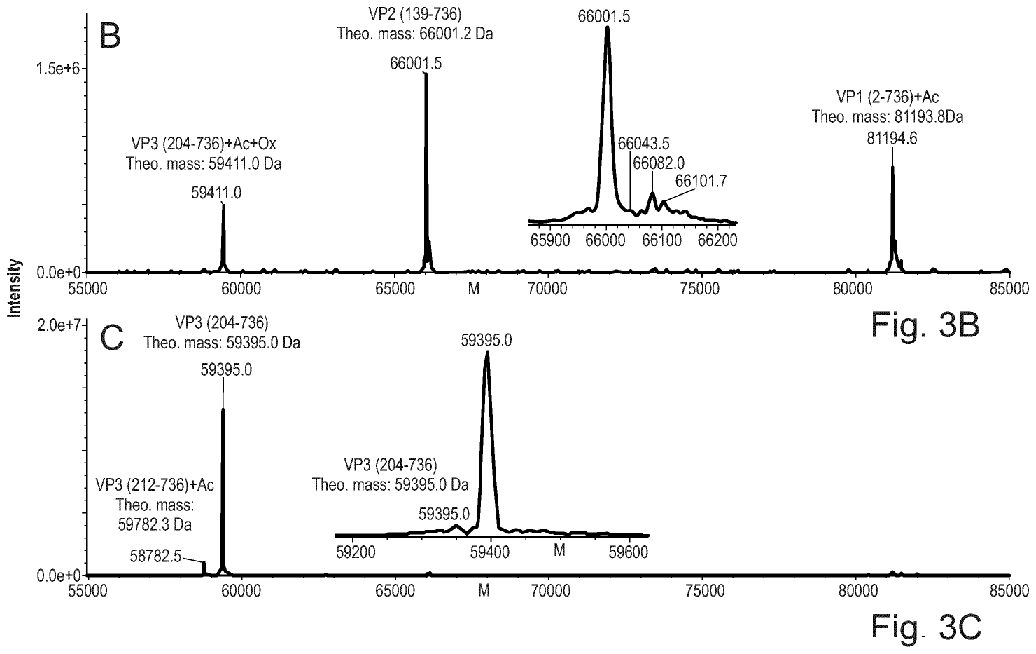

As the main aim was to develop a fast, efficient and robust procedure for preparing samples of VPs, intact mass analysis was applied in initial screening to detect all VP species present in the sample. As shown by the UV chromatogram in FIG. 3A, VP1 and VP2 eluted first, forming a shoulder of the main (VP3) peak. UV (280 nm) chromatogram (FIG. 3A) and deconvoluted mass spectra of the peaks eluting at the retention times of 46.5 min (FIG. 3B) and 47.2 min (FIG. 3C). Enlarged deconvoluted mass spectra from the strongest signals from each peak are displayed in the corresponding insets.

Four major peaks were detected with masses of 81194.6 Da, 66′001.5 Da, 59′411.0 Da, and 59,395.0 Da. The measured mass of 66,001.5 Da matches the amino acid sequence 139 to 736 of VP2, while the 81′194.6 Da and 59′395.0 Da masses correspond to amino acids 2 to 736 of VP1 and 204 to 736 of VP3, respectively, in each case with a 42 Da mass shift, indicating one acetylation on each VP.

The other main peak, with a mass of 59′411.0 Da, corresponds to an oxidized variant of the acetylated 204 to 736 sequence of VP3. In addition, a weak signal with 59′350.0 Da mass was detected, corresponding to amino acids 204 to 736 of VP3. Several low-intensity signals with different modifications of VP2 (acetylation and phosphorylation) were also detected. The theoretical mass of each VP was calculated, assuming that no reduced disulfide bonds were present as no evidence of disulfide linkages has been reported. The most plausible assignments of all detected signals are shown in Table 1.

TABLE 1
Experimental and theoretical masses of the viral
capsid proteins identified by the LC-MS analysis.

| | Amino Acid | | | |
|---|---|---|---|---|
| Capsid | Sequence and | Theoretical | Observed | Signal |
| Proteins | Modification | Mass (Da) | Mass (Da) | Intensity |
| VP3 Variant | VP3 (212 to 736) + Ac | 58′782.3 | 58′782.5 | Low |
| VP3 | VP3 (204 to 736) | 59′353.0 | 59′350.0 | Low |
| VP3 Variant | VP3 (204 to 736) + Ac | 59′395.0 | 59′395.0 | High |
| VP3 Variant | VP3 (204 to 736) + Ac + Ox | 59′411.0 | 59′411.0 | High |
| VP2 Variant | VP2 (139 to 736) | 66′001.2 | 66′001.5 | High |
| VP2 Variant | VP2 (139 to 736) + Ac | 66′043.2 | 66′043.5 | Low |
| VP2 Variant | VP2 (139 to 736) + Phos | 66′081.2 | 66′082.0 | Low |
| VP2 | VP2 (138 to 736) | 66′102.3 | 66′101.7 | Low |
| VP1 Variant | VP1 (2 to 736) + Ac | 81′193.8 | 81′194.6 | High |

Ac, Acetylation; Ox, Oxidation; Phos, Phosphorylation

### Peptide Mapping Analysis: Amino Acid Sequence Coverage

Multiple digestion strategies have been applied in LC-MS/MS analyses of VPs to ensure full coverage of their amino acid sequences, confirm the N- and C-terminal amino acid sequences, and both quantify and localize positions of PTMs. Trypsin digestion has not typically provided full sequence coverage due to the low frequencies of arginine (R) and lysine (K) in VP sequences, giving rise to long and hydrophobic tryptic peptides, or high frequency of R causing the generation of small hydrophilic tryptic peptides that might be missed in subsequent LC-MS/MS analysis.

Peptides generated by electrospray ionization following trypsin digestion mainly have multiple charges (≥2) due to their C-terminal amino acids (K and R). Therefore, the MS/MS spectral acquisition in this study was designed to detect ions with multiple charge states, as described in Material and Methods. In the VP1 amino acid sequence, three hydrophilic peptides could not be identified, so there were gaps in the amino acid sequence obtained. Tryptic peptide ANQQK (aa 34 to 38) was not detected in either replicate, while peptides TAPGK (aa 138 to 142) and peptide QQRVSK (QQR/VSK, aa 486 to 491) were not detected in one of the replicates. Manual searches for these short peptides confirmed their elution in flow-through of the reversed phase column (2.0 to 2.7 min), mainly in the form of singly charged ions. These ions were rejected for fragmentation during MS/MS acquisition and hence were not identified in the amino acid sequence of VP1 by Protein Metrics software due to lack of MS/MS information. Use of a column with lower particle pore size (130 Å instead of 300 Å) or a stronger ion pairing reagent like DFA in the mobile phases might enable better retention of these peptides on a reversed phase column.

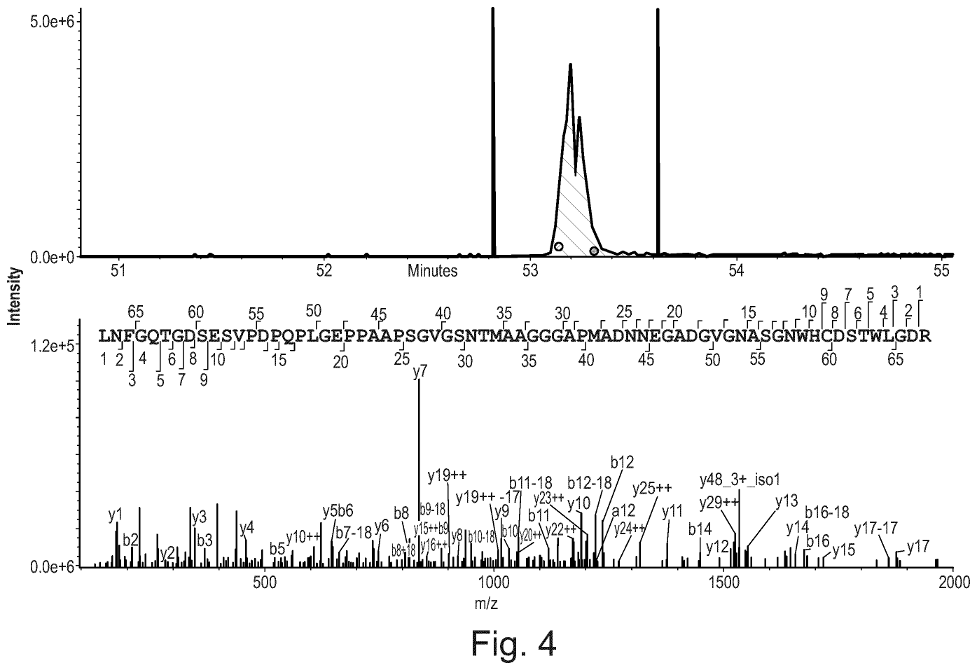

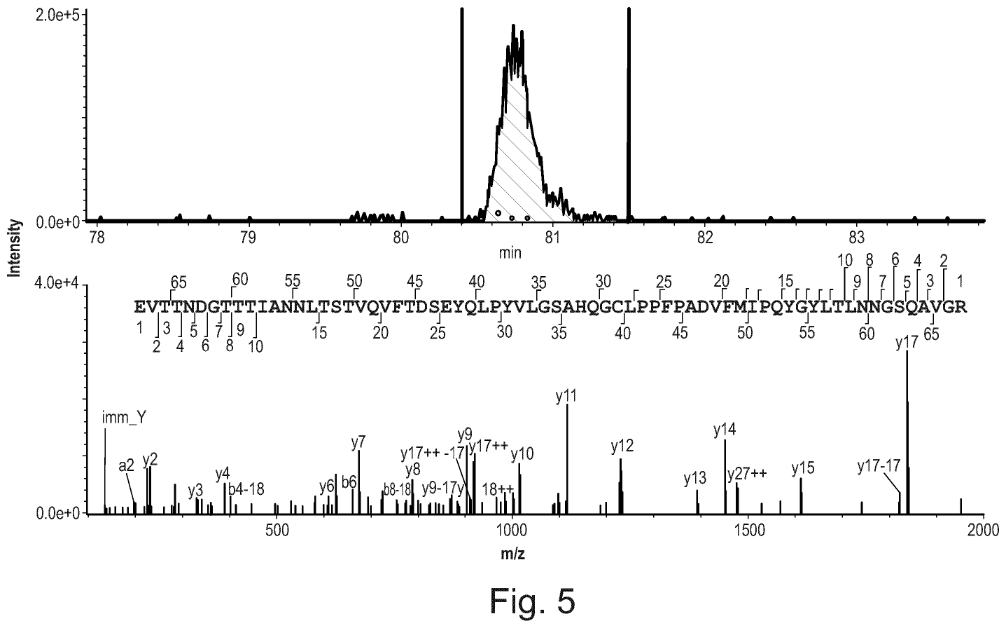

Next, detection and recovery issues of two large tryptic peptides (T23: aa 171 to 238 and T33: aa 323 to 390) generated by trypsin digestion of VP1 were investigated. These peptides are highly hydrophobic and therefore difficult to either keep in solution or elute from the reversed phase column. Both peptides were successfully detected with proper signal intensities. Extracted ion chromatograms (XIC) and MS/MS signals of T23 and T33 are presented in FIG. 4 and FIG. 5, respectively. Although the T33 peptide was so hydrophobic that it eluted just after the end of the 80 min 1 to 60% solvent B gradient (at min 81), no carry-over to the next run was observed.

All hydrophilic and hydrophobic peptides of VP1 were detected after trypsin digestion, and 100% sequence coverage by a single LC-MS/MS run was obtained. These observations clearly demonstrate that SDC/DDM enables simple, efficient trypsin digestion of VP.

To evaluate the compatibility of protein precipitation by Approach 1 with the current proteomics workflow, a protein pellet was subjected to Asp-N digestion and LC-MS/MS analysis, as described in Material and Methods. Searches of the resulting peptides against VP1 amino acid sequences indicated >97% sequence coverage.

Asp-N digestion also generates several short peptides due to high aspartate frequency in the VP1 amino acid sequence, which could lead to singly charged ions and hence gaps in the obtained VP1 amino acid sequence. Four such gaps were detected in the sequence obtained following Asp-N digestion. The N-terminal sequence (aa 1 to 12) includes several aspartates and can provide the following short peptides under optimal conditions: MAA (aa 1 to 3), DGYLP (aa 4 to 8) and DWLE (aa 9 to 12). The first methionine residue of cellular proteins is often cleaved off by methionine peptidase after protein synthesis, then the second amino acid residue becomes acetylated. This modification was confirmed by intact mass analysis (Table 1). However, neither this peptide (AA) nor other dipeptides (530 to 531 and 609 to 610) were detected by LC-MS/MS analysis following Asp-N digestion.

Manual searches for other amino acids that were missed in Asp-N digests (DGYLP, DWLE and DFAV) confirmed the presence of three singly charged ions that were strongly retained on the reversed phase column (retention times: 33.6, 35.0 and 32.2 min, respectively) due to high hydrophobicity. In total, six amino acids were not identified by the protocol involving Asp-N digestion, which thus provided 99.2% coverage. As trypsin digestion provided full sequence coverage, the main advantage of using additional Asp-N digestion is to identify MS/MS fragments that trypsin digestion misses (e.g. peptides T23 and T33).

### Characterization of N- and C-Terminal Sequences of VP1, VP2 and VP3

VP1, VP2, and VP3 share the same C-terminal region and only differ in their N-termini. The entire VP3 amino acid sequence (aa 203 to 736) is involuted in VP2 (aa 138 to 736), and the entire VP2 amino acid sequence is involuted in VP1 (aa 1 to 736). The N-terminus amino acid sequence and PTMs in VPs play important roles in the endosomal escape and cellular transport of viral particles. Therefore, in-depth characterization of VPs used as vectors in gene therapy (or other applications) is important not only to expand product knowledge but also to ensure product and process consistency.

The detection and identification of N- and C-terminal regions of VPs is provided to highlight the efficiency of the presented approach for detailed VP structural characterization.

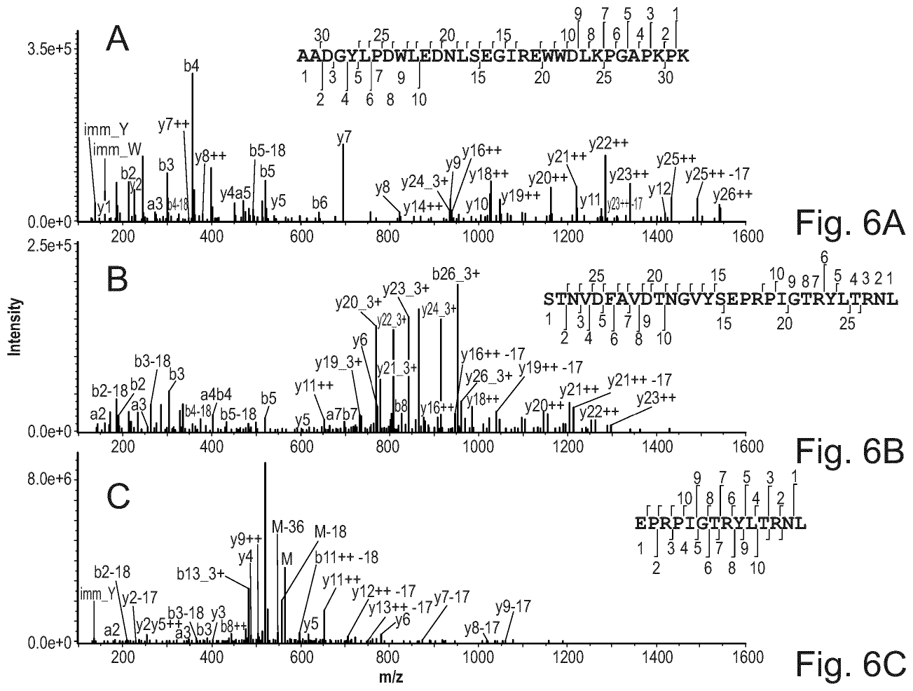

Amino acid sequences of both N- and C-terminal regions of VP1 were confirmed by the MS/MS spectra of tryptic peptides (FIGS. 6A and 6B), whereas Asp-N digest only provided this information for the C-terminus (FIG. 6C).

Acetylation of the first Alanine (A) in the N-terminal amino acid sequence of VP1 (FIG. 6A) was confirmed via the mass shift (+42 Da) observed in b-fragment ions (b2 to b6). In addition, detection of complete y ion series in the MS/MS spectrum of the Asp-N peptide (FIG. 6C) gives unambiguous confirmation of the amino acid sequence in the C-terminal peptide of VP1. However, this C-terminal peptide is shared in VP1, VP2 and VP3. This finding also confirms the assignment of VP1 by intact mass analysis as described in Table 1.

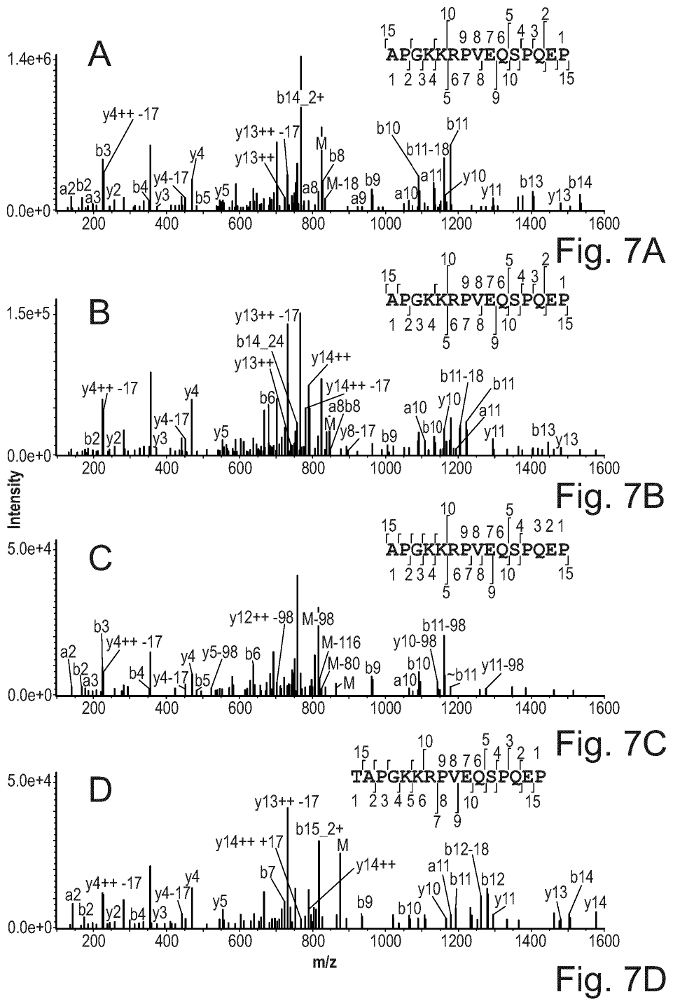

The main signal (66′001.5 Da) obtained from intact mass analysis of VP2 was assigned to amino acids 139 to 736, with low levels of acetylation and phosphorylation (Table 1). In addition, a weak signal at 66′101.7 Da was assigned to the full sequence of VP2 (138 to 736). To confirm these assignments, and both quantify and localize the modifications, peptide-mapping data were evaluated. Analysis of tryptic digests of VP2 revealed that its N-terminus starts with a threonine (TAPGK), while peptides with and without the threonine resulted from the Asp-N digestion. The reason for this inconsistency is that the tryptic peptide without threonine (APGK) produced a singly charged ion, and thus was not identified in sets of amino acid sequences, as already described. In contrast, both peptides were detected in analyses of Asp-N digests, and the peptide without threonine provided the main signal. In addition, when threonine was not present at the N-terminus, low levels of acetylation (0.3%) and phosphorylation (3.3%) of alanine and serine, respectively, were detected (FIGS. 7A to 7D) (N-terminus of VP2 without threonine APGKKRPVEQSPQEP (A), N-terminus of VP2 without threonine and with acetylation of the first alanine A (Ac) PGKKRPVEQSPQEP (B), N-terminus of VP2 without threonine and with phosphorylation of the serine APGKKRPVEQS (Phos) PQEP (C), and N-terminus of VP2 with threonine TAPGKKRPVEQSPQEP (D)). Phosphorylation in VP2 was identified via an observed neutral loss of 98 Da within the MS/MS spectra (FIG. 7C).

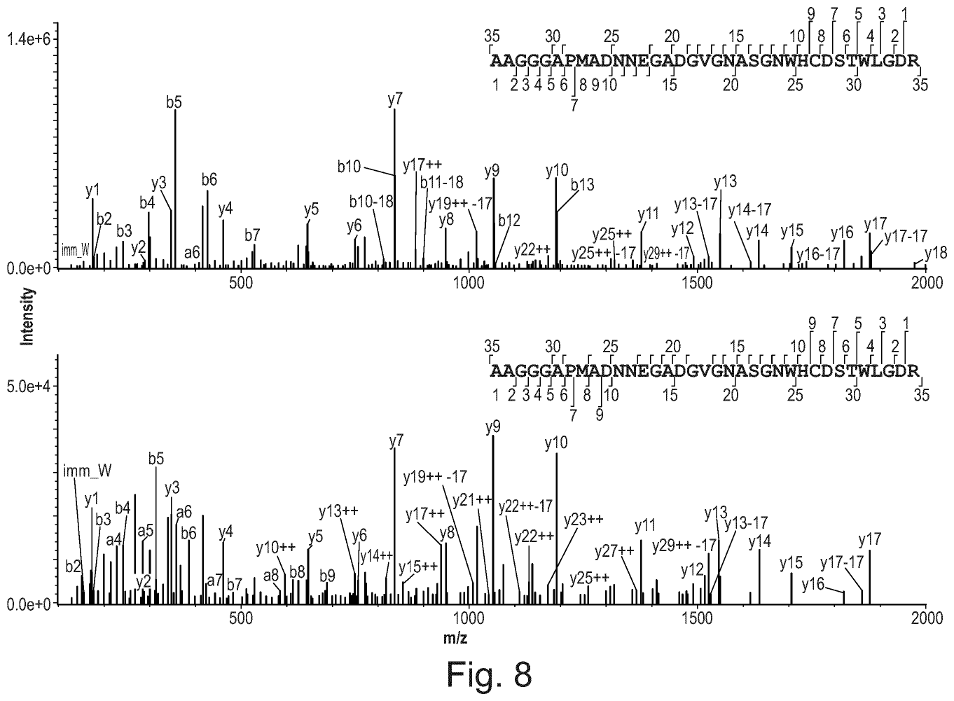

Similarly to VP1, the data showed that the methionine is cleaved from the N-terminus of VP3 and MS/MS data confirmed that in most cases the first alanine in the amino acid sequence was acetylated (FIG. 8). However, low levels of non-acetylated peptides were also detected (0.7%), as shown in FIG. 8. These data are consistent with results of the intact mass analysis of VP3 (Table 1). In addition, several PTMs such as deamidation, phosphorylation and methionine oxidation in VP1 were quantified by the present approach.

### Conclusion

The results provided herein clearly show that protein precipitation by methanol/water/chloroform is a robust and convenient method for isolating VPs from their complex matrices and other capsid impurities. The procedure is simple and applicable for various AAV-based vaccines and serotypes, regardless of the matrix type and sample quantity. It can also be easily adapted for use in different proteomic workflows involving different enzymatic digestions, and provide important complementary information, such as MS/MS fragmentation patterns and PTMs of VPs.

To circumvent issues in detection of long hydrophobic peptides, a trypsin digestion strategy based on SDC/DDM-enabled detection of long hydrophobic peptides was developed and achieved 100% VP1 amino acid sequence coverage with a single LC-MS/MS run. This approach significantly reduces sample processing steps and makes it accessible for a broad range of biological laboratories, with no need for high expertise in proteomic workflows. The workflow can also be applied without a second enzymatic digestion, making it advantageous when numerous samples must be rapidly characterized. Moreover, use of rapid and robust LC-MS/MS in combination with minor adjustments, e.g., optimization of digestion time, can further expand the method's capability for high-throughput analysis and accelerate both the development and processing of gene therapy products.

### Example 2: Extraction and Analysis of Adenovirus Capsid Proteins

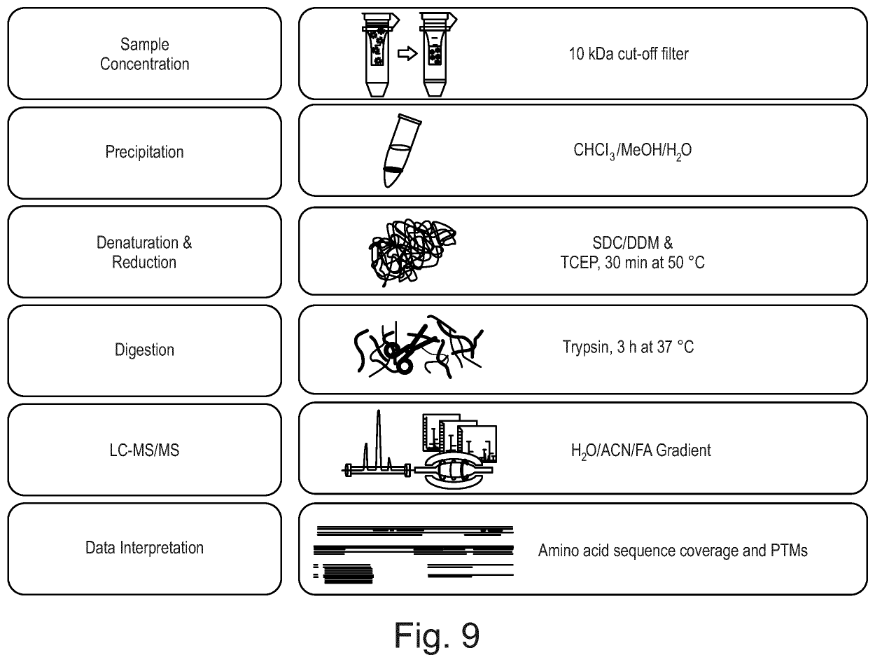

FIG. 9 shows an overview of the extraction and analysis protocols outlined in the following Example. The workflow involves concentration of AdV5 samples with an appropriate cut-off filter (10 kDa) to reduce their volume, followed by precipitation of VPs, their solubilization in SDC/DDM solution, reduction and digestion with trypsin. Finally, generated peptides are analyzed by LC-MS/MS. The method has two purposes: to confirm identities of the main VPs of AdV5 (high amino acid sequence coverage) and quantify their PTMs.

### Materials and Methods

### Chemicals

Tris 2-carboxyethyl phosphine (TCEP), ammonium bicarbonate (ABC), ultrapure formic acid, acetic acid, guanidine-HCl, chloroform, methanol, acetonitrile (ACN), water, trifluoroacetic acid (TFA) and sodium deoxycholate (SDC) were purchased from Sigma-Aldrich (St. Louis, MO). Vivaspin 500 (10 kDa MWCO) was purchased from Cytiva (Marlborough, MA). Sequencing grade trypsin and N-dodecyl-beta-D-maltoside (DDM) were purchased from Promega (Milwaukee, WI) and Thermo Fisher Scientific (Waltham, MA), respectively.

### Vector Production and Purification

One vial of HEK293 RCB was thawed and expanded in various sizes of shake flasks every 3 or 4 days. Once a sufficient cell mass was reached, the culture was used to inoculate a WAVE20 perfusion bioreactor at a target seeding density. The culture was medium exchanged and infected with wild type AdV5. The culture then was lysed and clarified with filters and stored as column load. The two batches of column loads were thawed and purified using a HR16 column packed with Source 15Q resin. The purified AdV5 virus particles were pooled and sterile filtered using a 0.2 μm filter. The filtered samples were aliquoted for LC-MS analysis.

### Sample Preparation

Samples of recombinant AdV5 were prepared as described below. A recombinant human monoclonal IgG4 antibody (designated mAb A) was also produced and purified at Lonza using standard manufacturing procedures.

Peptide mapping was performed after reducing the volume of 300 μL of AdV5 samples prepared as described above (containing roughly 40 μg of AdV5 VPs) to 200 μL before protein precipitation, as described below. The volumes were reduced by centrifuging samples over 10 kDa cut-off Vivaspin filters, also known as membrane filters, at 10′000 g and 20° C. for about 20 min.

### Protein Precipitation by Chloroform/Methanol/Water

VPs were precipitated with chloroform/methanol/water. Briefly, 800 μL of methanol, 200 μL of chloroform and 600 μL of water were sequentially added to 200 μL of concentrated sample (prepared as described above) with short vortex-mixing and fast centrifugation steps (10 sec at 14′000 g) following each addition. The protein precipitate appeared at the interface as a white layer between upper and lower phases. The upper phase was carefully removed and discarded, then 200 μL of cold methanol was added to the remaining mixture. After centrifugation, the supernatant was removed. Finally, the protein pellet was dried by vacuum centrifugation.

In all precipitation steps, cold solvents (4° C.) were used, and centrifugation was performed at 14′000 g and 4° C. for 10 min.

Dissolving with SDC and DDM and Enzymatic Digestion of mAB-a and Capsid Proteins

For trypsin digestion, Portions (60 μg) of mAb A were dissolved in 5 μL of a mixture of SDC (0.5, 0.75, 1.0, 1.25, 1.5, 1.75 and 2.0% w/v) and DDM (0.5% w/v) in 50 mM ammonium bicarbonate (pH 8.0). Then, following vortex-mixing at RT, the samples were diluted to 0.1, 0.15, 0.2, 0.25 0.3, 0.35 and 0.4% w/v of SDC and 0.1% w/v of DDM by adding by adding 20 μL of 50 mM ammonium bicarbonate (pH 8.0). Proteins were reduced by adding 1 μL of 500 mM TCEP and incubating the resulting mixtures for 30 min at 50° C. The samples were subjected to trypsin digestion (with a 30:1 w/w protein to protease ratio) for 3 hours at 37° C., then the digests were transferred directly to an HPLC vial for LC-MS/MS analysis, as described below.

Alternatively, viral protein pellets prepared as described above were dissolved in 5 μL of a mixture of SDC (1.75% w/w) and DDM (0.5% w/w) in 50 mM ammonium bicarbonate (pH 8.0). Then, following vortex-mixing at RT, the samples were diluted to SDC and DDM concentrations of 0.35% and 0.1% w/v, respectively, by adding 20 μL of 50 mM ammonium bicarbonate (pH 8.0). Proteins in the samples were reduced and digested as described above. The digests were then transferred directly to an HPLC vial for LC-MS/MS analysis, as described below.

### Liquid Chromatography-Mass Spectrometry

A system consisting of a Vanquish UPLC instrument coupled to an Orbitrap Fusion Lumos mass spectrometer (Thermo Fisher Scientific, Bremen, Germany) was used for all analyses. For peptide-mapping analysis an Acquity UPLC Peptide CSH C18 column (130 Å, 1.7 μm, 2.1 mm×150 mm, Waters Corporation) was used to separate peptides in the digested samples, with a mobile phase consisting of 0.1% v/v formic acid in water (A) and acetonitrile (B). The peptides were separated and eluted at a flow rate of 0.25 mL/min with a linear gradient from 1% v/v B to 30% v/v B over 140 min, followed by 30% v/v B to 40% v/v B over 15 min. The column was then washed with 98% v/v B for 10 min and conditioned with 1% v/v B for 8 min before the next injection.

The mass spectrometer was operated in data-dependent mode in the 200-2000 m/z range with a spray voltage of 3.5 kV and heated capillary temperature of 320° C. Full-scan spectra were recorded with a resolution of 120′000 (full width at half maximum resolution at 400 m/z) using an automatic gain control (AGC) target value of 2.0e5 with a maximum injection time of 100 ms. Up to 20 of the most intense ions with 2-8 charge states were selected for higher-energy c-trap dissociation (HCD) with a normalized collision energy of 35%. Fragment spectra were recorded at an isolation width of 2.5 Da and resolution of 15′000 using an AGC target value of 5.0e4 and maximum injection time of 200 ms. Dynamic exclusion was activated for 5 s within a 10 ppm window for precursor selection. Fragment ions were recorded by the orbitrap analyzer.

### Data Analysis

Raw mass spectral data were processed with Protein Metrics (San Carlos, CA). For peptide mapping, database searching against AdV5 viral capsid protein sequences was performed with tolerances of 6 and 20 ppm for peptides detected in MS and MS/MS analyses, respectively. For this, FASTA protein sequence files were downloaded from the UniProt database including the complete reviewed entries (31) of “human adenovirus C serotype 5”. N-terminal acetylation, methionine and tryptophan oxidation, phosphorylation, and asparagine deamidation were included as variable modifications in the searches.

### Results and Discussion

### Development of a Robust and Reproducible Sample Preparation Workflow

There have been strenuous efforts to develop a universal, robust and reproducible sample processing strategy for proteomic analysis. Important factors to consider when choosing procedures to minimize sample losses and maximize detection of peptides by MS include sample concentration and matrix type. However, there is little consensus regarding optimal sample processing protocols, and this step remains the main bottleneck for successful proteomic analysis. Sample losses during the processing steps are inevitable, which does not critically affect the performance of most methods if sufficiently large quantities of the starting materials (at least 50-150 μg of protein) are available. However, sample losses can pose major problems in proteomic analysis of gene therapy products (GTPs), as typically only limited amounts of materials are available during developmental stages (e.g., early clinical phases). The complex sample matrices and wide molecular weight ranges of VPs in GTPs compared to those in recombinant protein therapeutics also complicate their proteomic analysis.

Thus, it is challenging to apply general classical sample processing workflows for proteomic analysis of GTPs and a new approach is needed. Such an approach must be able to overcome these problems, efficiently remove interferences in sample matrices, and disassemble capsid proteins with no need for any further treatment. It should also involve minimum liquid-liquid handling steps, with no protein- or peptide-level clean-up step, and cause minimal artificial modification in sample processing to enable accurate quantification of PTMs. Moreover, it should require use of a single protease that can digest, and provide high amino acid sequence coverage of, all the VPs in the low amounts of AdV available in samples from early developmental steps.

Example 1 described a sample preparation method for proteomic analysis of adeno-associated viruses (AAVs) that circumvents many of the challenges associated with the common classical approach. The structural composition (e.g. capsid proteins and double-stranded DNA) and matrix complexity of AdVs are similar to those of AAVs, but the throughput of the method described in Example 1 could be limited by the low relative abundance of several components of the AdV5 proteome (0.1-0.3% of the total protein mass). Therefore, the AAV method required further optimization to reduce potential sample losses. Due to the limited available amounts of AdV5 materials, a monoclonal antibody (mAb A) was used initially for proof-of-concept in all optimization steps of the workflow, as described above.

The method essentially involves concentration of virus particles using a centrifugal filter with an appropriate cut-off membrane (10 kDa). This is followed by use of organic solvents to disassemble the particles into their structural proteins and DNA, with precipitation of the VPs at the interface between organic and aqueous phases. One of skill in the art will appreciate that the composition of organic solvent and aqueous phase may be adjusted to yield optimal results, depending on the protein of interest, sample matrix components and other factors. Sample matrix components and DNA partition into either the aqueous or organic phase and are removed prior to lyophilization of the protein precipitate. Importantly, in this new approach tertiary structures of the proteins are disrupted after precipitation and a further denaturation step with common chaotropic reagents (e.g. urea or guanidine hydrochloride) is thus not necessary.

In order to minimize the number of sample processing steps, precipitated proteins are re-dissolved in a low volume of aqueous SDC/DDM solution. Dissolving proteins in SDC or DDM solution can increase potential cleavage sites by trypsin and enhance hydrophobic peptides' solubility, thereby improving amino acid sequence coverage.

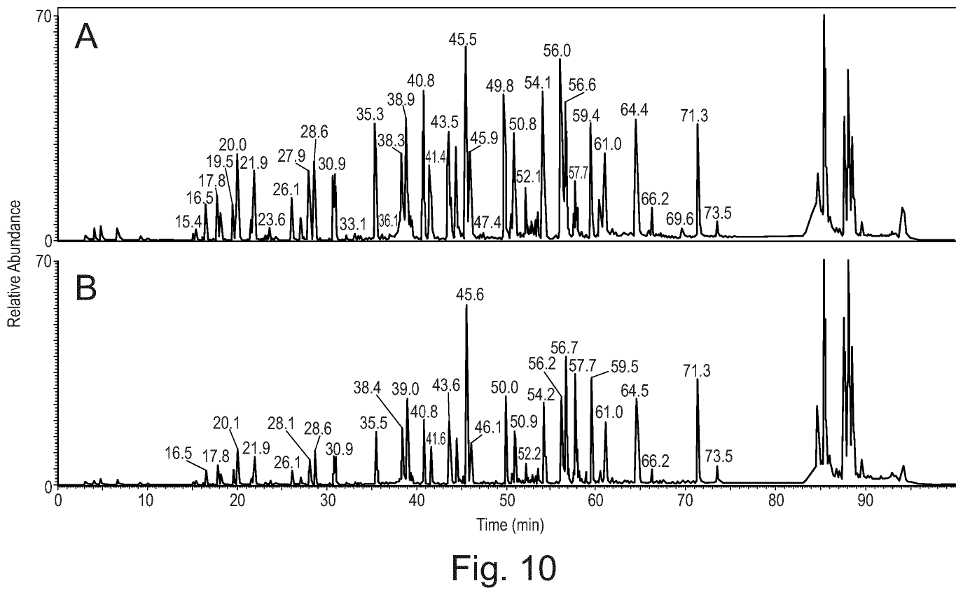

After digestion of the proteins it can be quite problematic to introduce a sample directly into an MS system, as a high concentration of SDC (1%) severely suppresses electrospray ionization and interferes with chromatographic separation of the peptides. Therefore, previously published protocols involve precipitation of SDC after addition of TFA and removal by centrifugation (i.e. acid centrifugation) or extraction with ethyl acetate (i.e. phase transfer). This step is a major bottleneck when low volumes of material are available. Under these conditions, the supernatant cannot be fully separated from the pellet, resulting in significant sample loss (up to 21%), as shown by results of precipitating SDC in mAb A solution, washing the pellet with water, and subjecting both supernatant as well as washed pellet to LC-MS/MS analysis (see FIG. 10). Washing the pellet with water or organic solvent and combining the wash solutions with the previously removed supernatant can improve the peptide recovery rate, but requires an additional sample processing step to decrease the large sample volume, e.g. by vacuum centrifugation, prior to LC-MS/MS. In the present study the workflow was further optimized by decreasing the percentage of SDC to enable omission of the SDC removal step and proceed directly to LC-MS/MS analysis.

To evaluate the possibility of direct LC-MS/MS analysis of the digested VPs, the following variables had to be assessed: solubility of SDC in an acidic mobile phase (precipitation risk), peak shapes and signal intensities of the peptides in the presence of SDC during chromatographic separation and MS detection, respectively, and the tryptic digestion of proteins in the presence of low concentrations of SDC.

Precipitation of SDC after injection of a digested sample into an acidic mobile phase was a likely scenario, which may lead to column blockage. Surprisingly, however, there was no evidence of SDC precipitation during incubation of mixtures of SDC/DDM with mobile phase A (with ratios varying from 1:1 to 1:10) at room temperature for 30 min. In addition, no increase in column pressure was observed during several successive LC-MS/MS analyses of digested mAB A samples (with and without SDC removal).

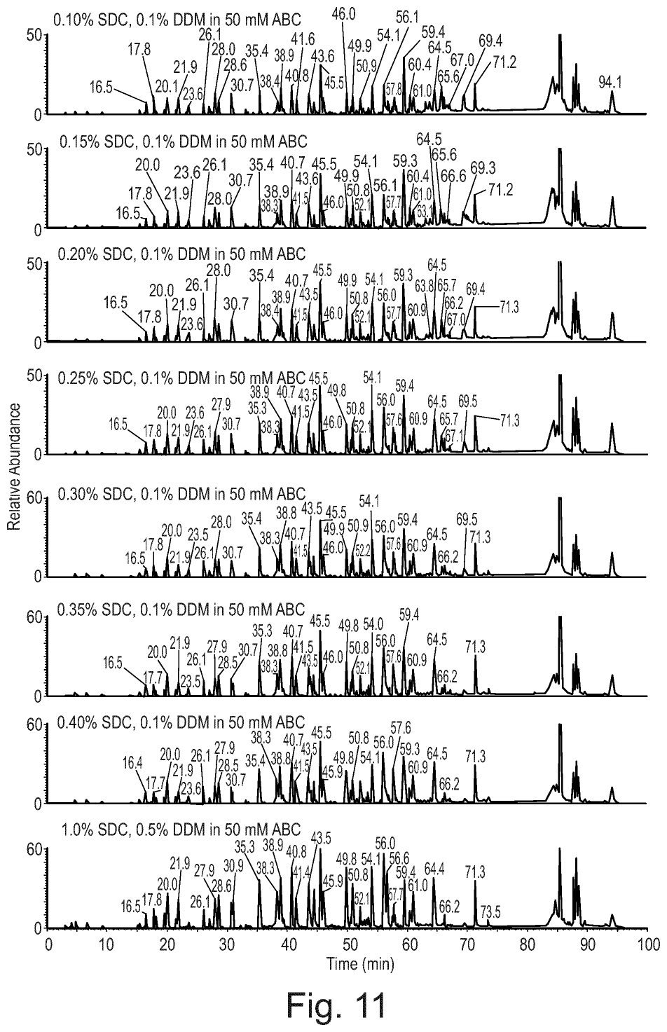

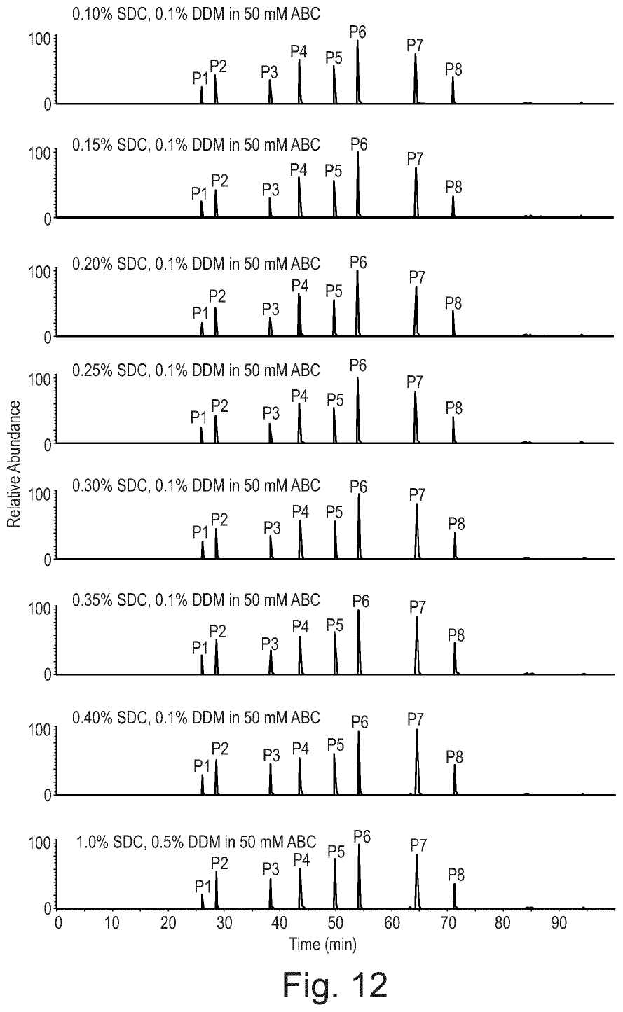

Chromatographic separation and MS detection were further evaluated by digesting mAb A in the presence of 0.1% w/v DDM and SDC at various concentrations (0.1, 0.15, 0.2, 0.25, 0.3, 0.35 and 0.4% w/v) then introducing the resulting peptides directly to the LC-MS/MS system, as described above. Signal intensities and peak shapes of eight identified peptides (here named P1 to P8) were compared to those obtained with the classical approach (digestion in 1% SDC followed by SDC precipitation and removal after addition of 2 μL TFA). The obtained data showed no differences in the chromatographic separation and peak shapes of the selected peptides across the entire gradient (see FIGS. 11 and 12). Signal intensities of the selected peptides tended to increase with increasing amounts of SDC up to 0.4% w/v (see Table 2).

TABLE 2
Signal intensity heights of the selected peptides of mAb A obtained in LC-
MS/MS analysis following processing with indicated percentages of SDC/DDM.

| | | 0.10% | 0.15% | 0.20% | 0.25% | 0.30% | 0.35% | 0.40% | 1.0% |
|---|---|---|---|---|---|---|---|---|---|
| | | SDC, | SDC, | SDC, | SDC, | SDC, | SDC, | SDC, | SDC, |
| | Peak | 0.1% | 0.1% | 0.1% | 0.1% | 0.1% | 0.1% | 0.1% | 0.5% |
| | name | DDM | DDM | DDM | DDM | DDM | DDM | DDM | DDM |
| Signal | P1 | 2.33E+8 | 2.78E+8 | 2.98E+8 | 3.75E+8 | 4.41E+8 | 4.95E+8 | 5.55E+8 | 2.53E+8 |
| Intensity | P2 | 3.64E+8 | 4.59E+8 | 5.83E+8 | 6.62E+8 | 7.69E+8 | 9.28E+8 | 9.66E+8 | 8.41E+8 |
| Height | P3 | 3.12E+8 | 3.28E+8 | 3.75E+8 | 4.53E+8 | 5.74E+8 | 6.60E+8 | 8.68E+8 | 8.45E+8 |
| | P4 | 5.72E+8 | 6.70E+8 | 8.49E+8 | 9.22E+8 | 9.55E+8 | 1.00E+9 | 1.01E+9 | 8.52E+8 |
| | P5 | 5.13E+8 | 6.17E+8 | 7.37E+8 | 8.60E+8 | 9.53E+8 | 1.12E+9 | 1.13E+9 | 1.12E+9 |
| | P6 | 8.30E+8 | 1.10E+9 | 1.33E+9 | 1.51E+9 | 1.64E+9 | 1.72E+9 | 1.75E+9 | 1.28E+9 |
| | P7 | 6.30E+8 | 8.04E+8 | 9.89E+8 | 1.16E+9 | 1.37E+9 | 1.53E+9 | 1.77E+9 | 5.19E+8 |
| | P8 | 3.57E+8 | 3.89E+8 | 4.99E+8 | 6.08E+8 | 6.76E+8 | 8.41E+8 | 8.52E+8 | 4.83E+7 |

It has been found that higher amounts of SDC can enhance the denaturation/solubilization of proteins (here mAb A) and enhance the protease performance, but even at the lowest concentration tested, overall digestion was still remarkably efficient. Of note, signal intensities of the selected peptides in the digested samples were significantly higher than those obtained with the approach involving SDC removal for SDC concentrations ≥0.35% w/v. For P7 and P8, even signal intensities of samples with SDC ≥0.25% w/v were higher than in samples obtained after SDC removal (Table 2). This could be related to the hydrophobic nature of these peptides and partial precipitation that may occur during the SDC removal step. The signal intensities of the selected peptides with 0.35% and 0.4% w/v of SDC were comparable, but 0.35% w/v SDC was selected as the optimal concentration for further experiments to avoid potential contamination in the source of the mass spectrometer during long-term use.

In Example 1, DDM was used as a combinatorial detergent to increase the solubility of the hydrophobic peptides and avoid their precipitation after SDC removal. The SDC removal step was omitted in the workflow presented here, but due to the lower SDC proportion (0.35% w/v) than in the standard workflow (1% w/v), DDM was added to enhance the proteins' solubility and hence increase the performance of the protease. To avoid mass overloading of the RP column after injection of all digested materials, the effect of lower levels of DDM (0.1, 0.2, 0.3, 0.4 and 0.5% w/v) on signal intensities of the selected peptides was evaluated. The results showed that similar signal intensities were obtained with all these DDM concentrations (data not shown), so 0.1% w/v DDM was utilized in further experiments. In summary, a combination of SDC (0.35% w/v) and DDM (0.1% w/v) in 50 mM ammonium bicarbonate (pH 8.0) solution was found to be the optimal buffer to dissolve and digest VPs of AdV5 for the characterization study.

The results obtained for mAB A demonstrate that the method described herein is useful for a range of proteins, including but not limited to AAV/AdV VPs. It should also be noted that the chosen model compound mAb A has a more rigid structure than VPs due to several disulfide bridges, so even lower concentrations of SDC could be sufficient for effective denaturation and solubilization of VPs.

### Amino Acid Sequence Coverage of AdV5 Capsid Proteins

Digested VPs of AdV5 were analyzed according to the developed method, as illustrated in FIG. 9 and briefly described above. Due to the vast differences in relative abundances of AdV5 structural proteins (e.g. Hexon and pTP account for 59.5 and 0.1% of the total protein mass, respectively), several modifications of the LC-MS/MS setup were required. First, in silico trypsin digestion of VPs of AdV5 resulted in generation of more than 350 peptides with four or more amino acids, so a longer gradient was needed to separate and identify the peptides. The highest sequence coverage was obtained when a longer gradient (150 to 180 min rather than a 100 min) was used. Additionally, to enhance retention and detection of small peptides, the column with 300 Å pores as used in Example 1 was replaced with one with smaller pores (130 Å).

Average amino acid sequence coverages (two technical replicates) for the major, cement and core proteins of 93.2, 97.7 and 85.0%, respectively, were obtained (see Table 3). The highest sequence coverages (≥99%) were obtained for Penton, pIIIa and pIX proteins, and the lowest (55%) for pTP. The sequence coverage of pTP was also lowest in a previous study (36% with trypsin digestion and 55% when three different proteases were used). These results show that obtaining high sequence coverage of pTP is very challenging. There are several reasons for this. First, it accounts for the lowest percentage (0.1%) of the total mass of VPs of AdV5, so it is close to the detection limit of the applied UHPLC-MS/MS system. Second, covalent attachment of pTP to DNA could complicate its isolation by the presented approach. However, precipitation by chloroform/methanol/water can generally remove contaminating DNA, so it was hypothesized that either pTP is not fully precipitated or precipitation of pTP with DNA could reduce trypsin's ability to digest it properly. Use of a nano-LC-MS/MS system and/or treatment with DNAase in the sample processing step could be used to further enhance pTP sequence coverage. Despite these complications, 55% sequence coverage of pTP was obtained, clearly showing that the presented approach is a convenient method for characterization of VPs with low abundance in complex matrices.

TABLE 3
Sequence coverage of the main proteins of AdV5.

| | | | | | | | Sequence |
|---|---|---|---|---|---|---|---|
| | | Uniprot ID | No. Residues (Mw) | | # Copies/virion* | | Coverage** (%) |
| Major | Hexon | P04133 | 952 | (108.0 kDa) | 720 | (240 trimers) | 91.4 |
| Proteins | Penton base | P12538 | 571 | (63.3 kDa) | 60 | (12 pentamers) | 98.9 |
| | Fiber | P11818 | 581 | (61.6 kDa) | 36 | (12 trimers) | 89.2 |

| | | | | | | |
|---|---|---|---|---|---|---|
| Cement | pIIIa | P12537 | 585 | (65.2 kDa) | 68 ± 2 | 99.1 |
| proteins | pVI | P24937 | 250 | (27.0 kDa) | 342 ± 4 | 98.6 |
| | pVIII | P24936 | 227 | (24.7 kDa) | 127 ± 3 | 93.8 |
| | pIX | P03281 | 140 | (14.5 kDa) | 247 ± 2 | 99.3 |
| Core | pV | P24938 | 368 | (41.4 kDa) | 157 ± 1 | 94.3 |
| Proteins | pVII | P68951 | 198 | (22.0 kDa) | 833 ± 19 | 91.9 |
| | pTP | P04499 | 671 | (76.5 kDa) | 2 | 55.1 |
| | pμ | Q2KS10 | 80 | (8.8 kDa) | ~104 | 92.5 |
| | AVP | P03253 | 204 | (23.1 kDa) | 15 ± 5 | 91.7 |
| | pIVa2 | P03271 | 449 | (50.9 kDa) | 7 ± 1 | 84.2 |

*Literature values;
**Average of two replicate.

Lower sequence coverages for some proteins of AdV5 (e.g. pV and pVII) are related to generation of small peptides by trypsin digestion that are not retained on the RP column. These proteins have high frequencies of arginine and lysine in their amino acid sequences and the resulting tryptic peptides (1-3 amino acids) cannot be retained even by a column with small (130 Å) pores. However, 94 and 92% amino acid sequence coverage for pV and pVII, respectively were still obtained in this study.

A recent LC-MS/MS-based study confirmed the presence of additional VPs, classified as ‘non-structural proteins’, which might be present in AdV5 virus particles. To further investigate the VPs, peptides identified from the MS/MS data were used in searches against a list of human adenovirus C serotype 5 proteins in the UniProt database (31 reviewed proteins) and an additional 14 non-structural viral proteins were found using the approach presented here. They are expected to have low abundance compared to structural viral proteins of AdV5 and their presence could be related to the type of purification steps used. To our knowledge, there is insufficient information regarding details and roles of these proteins in virus infections. However, use of an LC-MS/MS system with a lower flow rate and higher sensitivity (e.g. a micro- or nano-LC-MS/MS system) could further enhance their detection and identification if necessary.

Amino acid sequences of some main structural proteins can differ among AdV serotypes, and peptide mapping analysis can be used for their identification. For instance, Hexon and Fiber proteins of AdV5 and AdV2 significantly vary and can be used as markers for serotype identification. In contrast, other VPs have much more constant amino acid sequences (e.g. pVII and pu in AdV5 and AdV2).

The obtained data show that the method described herein greatly increased the amino acid sequence coverage of the main structural proteins and can discriminate between different adenovirus serotypes, so it can be used to confirm identities of viral vectors.

### Quantitative Analysis of PTMs of AdV5

Quantifying PTMs of the VPs at site-specific level during the progression of an infection can provide important new information on the biological and pathogenetic mechanisms of viral infections. So far, limited information on these mechanisms has been obtained from quantitative proteomic approaches, and there is a need for further characterization of proteins in model viral systems like AdV5. The results of this study highlight the sensitivity of the presented approach for identifying multiple PTMs in a single LC-MS/MS run, and hence its potential application for correlating PTMs with biological functions. The analysis revealed a total of 53 PTMs in the main structural VPs of AdV5 (with at least 0.5% relative abundance), including phosphorylations, acetylations, deamidations and oxidations (see Table 4). These modifications are known to effect properties of GTPs (e.g. stability) and are influenced by multiple variables of the manufacturing process, including the cell line and purification techniques.

These modifications are discussed below with an example of an MS/MS spectrum. PTMs of the viral proteins are known from intra-cellular maturation of capsid proteins, but some of these modifications (e.g. deamidations and oxidations) can also be induced during purification, storage or sample processing in the proteomic workflow. Analysis of several therapeutic proteins by the presented approach confirmed that the applied workflow is unlikely to increase deamidation and oxidation levels (data not shown).

TABLE 4
Summary of PTMs of AdV5 proteins with ≥ 0.5% relative abundance. Only PTMs
with a relative abundance of ≥ 0.5% are listed.

| | | Peptide Sequence (mod. | | Mass error | |
|---|---|---|---|---|---|
| Protein | Modification | pos. underlined) | Mod. AA | (ppm) | % PTM |
| Hexon | Acetylation | ATPSMMPQWSYMHIS | A2 | 0.4 | 97.4 |
| | | GQDASEYLSPGLVQFA | | | |
| | | R | | | |
| | Acetylation / | ATPSMMPQWSYMHIS | A2, M6 | 0.2 | 0.8 |
| | Oxidation | GQDASEYLSPGLVQFA | | | |
| | | R | | | |
| | | ATPSMMPQWSYMHIS | A2, W10 | 0.4 | 0.5 |
| | | GQDASEYLSPGLVQFA | | | |
| | | R | | | |
| | Oxidation | VVLYSEDVDIETPDTHI | M303 | 0.6 | 0.7 |
| | | SYMPTIK | | | |
| | | VGNNFAMEINLNANL | M460 | 0.4 | 0.6 |
| | | WR | | | |
| | Deamidation | ATETYFSLNNK | N42 | 0.4 | 1.9 |
| | | TTPMKPCYGSYAKPT | N244 | 0.5 | 19.4 |
| | | NENGGQGILVK | | | |
| | | IIENHGTEDELPNYCFP | N403 | 0.8 | 0.9 |
| | | LGGVINTETLTK | | | |
| | | TGQENGWEK | N437 | 0.4 | 20.2 |
| | | VGNNFAMEINLNANL | N456 | 0.5 | 2.1 |
| | | WR | | | |
| | | WSLDYMDNVNPFNHH | N536 | 0.4 | 0.5 |
| | | R | | | |
| | | SMLLGNGR | N552 | 0.1 | 15.5 |
| | | YKDYQQVGILHQHNN | N825 | 0.9 | 0.6 |
| | | SGFVGYLAPTMR | | | |
| | Phosphorylation | ATPSMMPQWSYMHIS | S5 | 1.9 | 1.3 |
| | | GQDASEYLSPGLVQFA | | | |
| | | R | | | |
| Penton | Deamidation | QNGVLESDIGVK | N199 | 0.2 | 7.7 |
| | | SFYNDQAVYSQLIR | N477 | 0.8 | 0.9 |
| | | NSIGGVQR | N533 | 0.4 | 1.9 |
| Fiber | Deamidation | ARPSEDTFNPVYPYDT | N37 | 0.7 | 13.8 |
| | | ETGPPTVPFLTPPFVSP | | | |
| | | NGFQESPPGVLSLR | | | |
| | | LSEPLVTSNGMLALK | N59 | 0.4 | 14.3 |
| | | MGNGLSLDEAGNLTS | N68 | 0.5 | 17.3 |
| | | QNVTTVSPPLKK | | | |
| | | EPIYTQNGK | N200 | 0.6 | 17.4 |
| | | YGAPLHVTDDLNTLT | N231 | 0.4 | 1.7 |
| | | VATGPGVTINNTSLQT | | | |
| | | K | | | |
| | | NGDLTEGTAYTNAVG | N482 | 0.5 | 11.3 |
| | | FMPNLSAYPK | | | |
| | | SNIVSQVYLNGDK | N523 | 0.3 | 15.9 |
| | Phosphorylation | TKSNINLEISAPLTVTS | S102 | 0.3 | 22.1 |
| | | EALTVAAAAPLMVAG | | | |
| | | NTLTMQSQAPLTVHD | | | |
| | | SK | | | |
| pIIIa | Acetylation | MMQDATDPAVR | M1 | 0.2 | 100.0 |
| | | MQDATDPAVR | M2 | 0.1 | 78.0 |
| | Oxidation | LMVTETPQSEVYQSGP | M171 | 0.4 | 0.5 |
| | | DYFFQTSR | | | |
| | Deamidation | AALQSQPSGLNSTDD | N22 | 0.1 | 2.3 |
| | | WR | | | |
| | | LLGEEEYLNNSLLQPQ | N493 | 1.1 | 0.8 |
| | | R | | | |
| | | NLPPAFPNNGIESLVD | N510 | 0.6 | 4.1 |
| | | K | | | |
| | | NLPPAFPNNGIESLVD | N511 | 0.4 | 17.5 |
| | | K | | | |
| | Phosphorylation | RPSSLSDLGAAAPR | S450 | 0.2 | 3.2 |
| pVI | Acetylation | MEDINFASLAPR | M1 | 0.2 | 98.3 |
| | Acetylation / | MEDINFASLAPR | M1, M1 | 0.3 | 1.7 |
| | Oxidation | | | | |
| | Oxidation | AWNSSTGQMLR | M66 | 0.2 | 0.8 |
| | Deamidation | AWNSSTGQMLR | N60 | 0.7 | 2.3 |
| pVIII | Acetylation | SKEIPTPYMWSYQPQ | S2 | 0.6 | 100.0 |
| | | MGLAAGAAQDYSTR | | | |
| | Oxidation | EIPTPYMWSYQPQMG | W11 | 0.6 | 1.2 |
| | | LAAGAAQDYSTR | | | |
| | | INYMSAGPHMISR | M34 | 0.4 | 0.5 |
| | Deamidation | NNLNPR | N67 | 0.1 | 2.8 |
| | Phosphorylation | VRSPGQGITHLTIR | S118 | 0.4 | 19.9 |
| pIX | Acetylation | STNSFDGSIVSSYLTTR | S2 | 0.5 | 98.2 |
| | Oxidation | QNVMGSSIDGRPVLPA | M30 | 0.5 | 1.0 |
| | | NSTTLTYETVSGTPLE | | | |
| | | TAASAAASAAAATAR | | | |
| | Deamidation | QNVMGSSIDGRPVLPA | N43 | 0.7 | 1.7 |
| | | NSTTLTYETVSGTPLE | | | |
| | | TAASAAASAAAATAR | | | |
| | Phosphorylation | STNSFDGSIVSSYLTTR | S2 | 1.4 | 1.8 |
| pV | Oxidation | HKDMLALPLDEGNPT | M126 | 0.4 | 0.7 |
| | | PSLKPVTLQQVLPALA | | | |
| | | PSEEKR | | | |
| | | ESGDLAPTVQLMVPK | M175 | 0.4 | 0.7 |
| | | QVAPGLGVQTVDVQIP | M235 | 0.8 | 0.8 |
| | | TTSSTSIATATEGMET | | | |
| | | QTSPVASAVADAAVQ | | | |
| | | AVAAAASK | | | |
| | Phosphorylation | QVAPGLGVQTVDVQIP | S224 | 0.7 | 1.6 |
| | | TTSSTSIATATEGMET | | | |
| | | QTSPVASAVADAAVQ | | | |
| | | AVAAAASK | | | |
| pVII | Acetylation | SILISPSNNTGWGLR | S2 | 0.5 | 100.0 |
| | Deamidation | NYTPTPPPVSTVDAAI | N70 | 0.5 | 0.5 |
| | | QTVVR | | | |
| | Phosphorylation | NYTPTPPPVSTVDAAI | S79 | 0.4 | 0.9 |
| | | QTVVR | | | |
| pμ | Oxidation | MRGGILPLLIPLIAAAI | M48 | 0.4 | 5.9 |
| | | GAVPGIASVALQAQR | | | |
| | | H | | | |

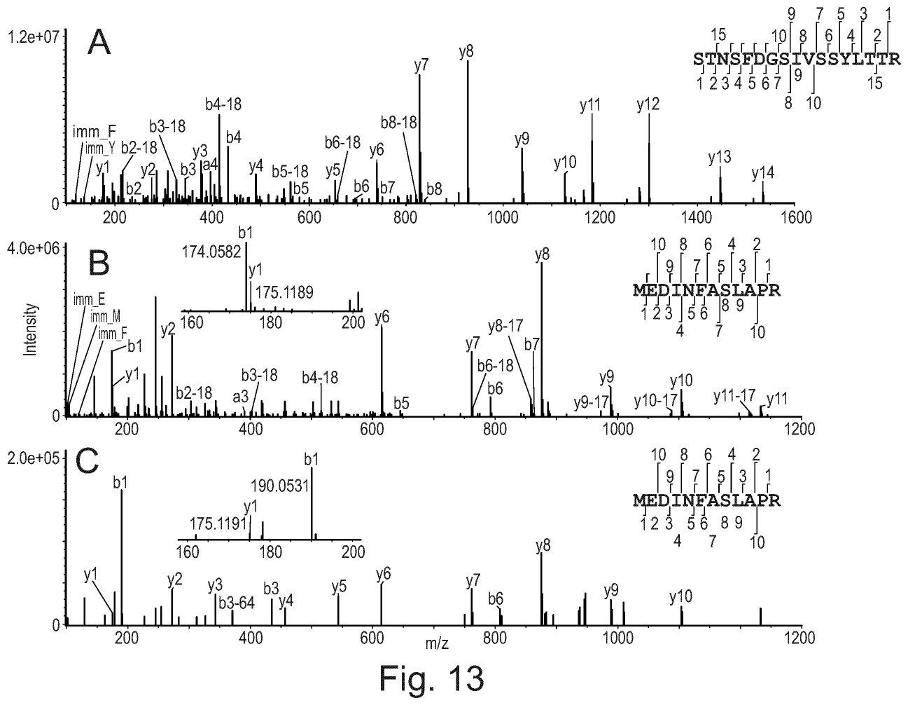

Protein acetylation is recognized as an important regulatory event during diverse infections with human viruses. Acetylation of the N-terminal amino acid is the most frequently detected type of modification of a given amino acid of the VPs. It has been shown that N-terminal acetylation of VPs can play a significant role in their intracellular trafficking and entry into the nuclei. Therefore, it is important to confirm their N-terminal sequences and quantify their acetylation in GTPs. This modification is confirmed by the associated mass shift (+42.01 Da) in total peptide mass, and its localization is confirmed by data on the b-fragment ions generated in the MS/MS analysis. Acetylation mainly occurs when methionine of the N-terminus of VPs is cleaved off by methionine aminopeptidase, then the resulting N-terminal amino acid residue is acetylated. For instance, it was found in the present study that ≥98.0% of N-termini of pIX were acetylated after methionine removal (FIG. 13A). The MS/MS spectrum of the corresponding peptide showed a series of b-fragment ions (b1-b8) with +42.01 Da mass shifts on the serine residue, confirming acetylation at the N-terminus. However, in the absence of N-terminal methionine removal, the retained methionine can also be acetylated, as demonstrated by a series of b-fragment ions (b1-b7) for the N-terminus of pVI with 42.01 Da mass shifts (FIG. 13B). Some of the amino acids can undergo additional modifications, for example, the N-terminal acetylated methionine of pVI could also be oxidized. This modification was confirmed by MS/MS analysis of the b1-fragment ions, as shown in the enlarged mass spectra in FIGS. 13B and C.

Acetylation at the N-termini of some VPs (e.g. Pentone base and Fiber) cannot be quantified by the approach presented here, as these proteins have high arginine and lysine frequencies in their N-terminal amino acid sequences, so these peptides can also not be retained and detected, as discussed above. However, use of a second protease (e.g. Asp-N) may provide the necessary information (cf. Example 1).

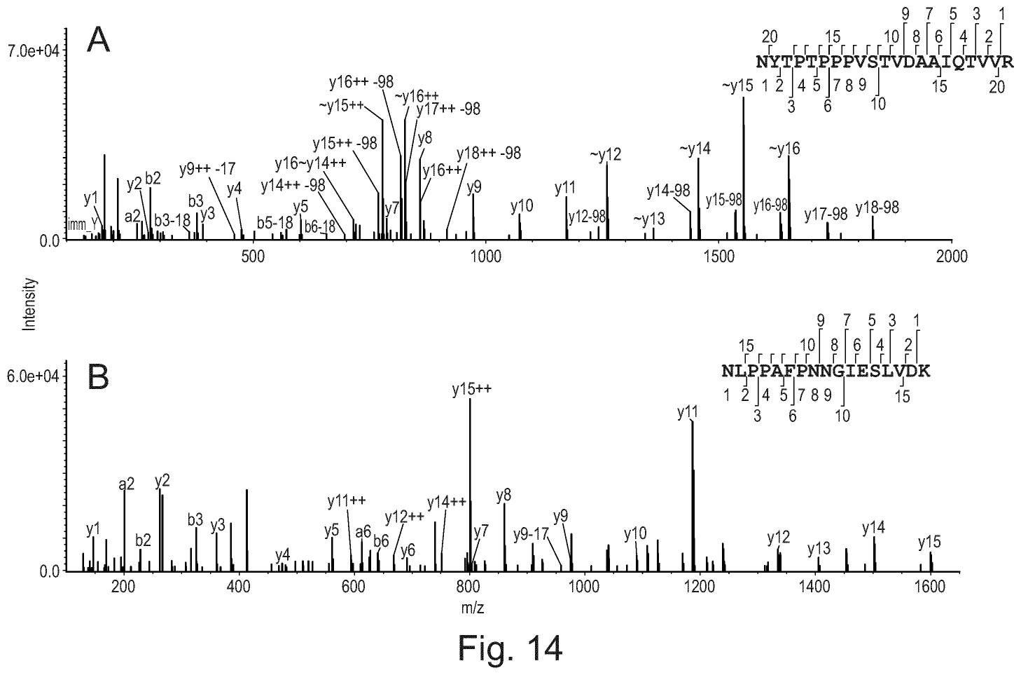

Phosphorylation of serine, threonine and tyrosine is another important type of PTM, which is involved in stability of virus capsids, and thus likely affects the infection process. Phosphorylation is identified by neutral loss of H₃PO₄ (97.98 Da) from proteolytic peptide molecular ions in MS/MS spectra (FIG. 14A). Relative abundances of phosphotyrosine (pY), phosphothreonine (pT), and phosphoserine (pS) in normally growing cells are approximately 2, 12, and 86%, respectively. The phosphorylated peptides have low intensities and for comprehensive analysis an additional chromatographic fractionation and enrichment step of the phosphopeptides is needed before LC-MS/MS analysis. Since such steps were absent in the present workflow, a lower number of the phosphorylation sites than in previous studies was observed. In total, seven serine phosphorylation sites and no tyrosine or threonine phosphorylation sites were identified, in accordance with their expected stoichiometric ratios.

Oxidation and deamidation are common PTMs in biopharmaceuticals, and they affect both biological activity and efficacy. These modifications are expected to be critical quality attributes (CQA) and therefore need to be evaluated throughout the development of viral vectors as pharmaceutical products. Nevertheless, effects of these protein modifications on the efficacy and safety of viral vectors have been poorly studied, despite their potential importance. For example, deamidation of amino acids on the surface of AAV capsids reportedly leads to charge heterogeneity and changes in vector functions. Effects of oxidation and deamidation on the stability and properties of virus particles can be simulated by appropriate exposure to hydrogen peroxide and solutions with high pH, respectively. Chemical modification in virus particles can be monitored by several methods, e.g. capillary zone electrophoresis (CZE), dynamic light scattering (DLS) and electrophoretic light scattering (ELS). However, these methods only provide holistic information on the virus particle level, and cannot quantify or localize these modifications on the protein or amino acid level.

RP chromatography is the only currently available method that can provide information on levels of oxidation in different VPs, through changes in protein retention times or appearance of new peaks, but it cannot localize these modifications in specific methionines or tryptophans of the VPs.

Using the novel approach presented here, 12 oxidation sites were identified in total in the main VPs of AdV5. Levels of oxidation were less than 1% at all these sites except M48 in pu (5.9%).

Deamidation of asparagine residues (resulting in a +0.98 Da mass shift) is a common, irreversible modification, and its mechanism has been studied by LC-MS/MS in great detail. It has been shown that deamidation rates depend on primary and higher-order structures of the proteins, pH and temperature. In addition, asparagine in SNG, ENN, LNG, and LNN amino acid motifs is reportedly most prone to deamidation. An example of an MS/MS spectrum of the corresponding deamidated peptides showed a series of y 10-yl5 fragment ions with a +0.98 Da mass shift on the corresponding asparagine's (FIG. 14B). As sample processing and digestion times are short in the presented workflow, the detected deamidation level (cf. Table 4) is highly unlikely to be artefactual. This was confirmed by detection of low levels of deamidation in mAb A during the method optimization steps, as described above. In total, 25 deamidation sites with mainly NG, NN and NS motifs in the main VPs of AdV5 were detected (Table 4). The highest level of deamidation in the VPs was associated with the NG motif. A similar study found that the main deamidations of AAV8 are in hypervariable regions (HVRs) with an NG motif. HVRs are largely responsible for interactions with target cells and the immune system, so they play a significant role in decreasing transduction by altering receptor binding.

As discussed above, the method presented here enables simultaneous quantification of multiple PTMs. Its applicability could be extended by changing the search parameters as appropriate for quantification of other PTMs (e.g. N- and O-glycosylation), if necessary. However, even without such enhancement it can provide more information than previous approaches about amino acid sequences and associated PTMs, thereby improving manufacturing processes of GTPs.

### Conclusion

The novel workflow for AdV VP analysis presented here affords substantially lower analysis times than traditional peptide mapping methods, which require use of several proteases and extensive sample processing for sufficient in-depth characterization. We provide here the first demonstration that a solution containing SDC and DDM can be used for simultaneous VP denaturation and digestion prior to direct LC-MS/MS analysis without any further cleanup steps. Surprisingly, despite the lower detergent concentration used for solubilization of the precipitate, analysis of the main VPs by the developed approach enabled substantially higher than previous average sequence coverage (up to 92%) as well as the quantification of 53 PTMs with single LC-MS/MS runs.

Along with increased analytical performance, the minimization of sample preparation steps reduces the risk of protein or peptide losses, and enables high-throughput analysis when numerous samples must be rapidly characterized to support the development, stability testing, and characterization of GTPs.

The comprehensive information provided by this approach can provide highly valuable mechanistic insights into viral infection that are difficult to obtain by other approaches. In addition, it is envisaged that the approach will motivate future research to monitor and control the host cell proteins of GTPs produced using different vectors and manufacturing processes.

### Exemplary Embodiments

Embodiment 1 is a method of preparing a digested virus protein, comprising: precipitating a virus protein from a sample containing the virus protein; dissolving the virus protein in a mixture comprising sodium deoxycholate (SDC) and N-dodecyl-beta-D-Maltoside (DDM) to generate a solution; and digesting the virus protein with a protease.
Embodiment 2 is a method of analyzing a digested virus protein, comprising: precipitating a virus protein from a sample containing the virus protein; dissolving the virus protein in a mixture comprising sodium deoxycholate (SDC) and N-dodecyl-beta-D-Maltoside (DDM) to generate a solution; digesting the virus protein with a protease; and analyzing the digested virus protein via liquid chromatography-tandem mass spectrometry (LC-MS/MS).
Embodiment 3 is a method of analyzing a digested virus protein, comprising: precipitating a virus protein from a sample containing the virus protein; dissolving the virus protein in a mixture comprising sodium deoxycholate (SDC) and N-dodecyl-beta-D-Maltoside (DDM) to generate a solution; digesting the virus protein with a protease; removing the SDC from the solution; and analyzing the digested virus protein via liquid chromatography-tandem mass spectrometry (LC-MS/MS).
Embodiment 4 includes the method of any one of Embodiments 1 to 3, wherein the virus protein is an adeno-associated virus capsid protein (AAV capsid protein), an adenovirus protein, a lentivirus protein, a retrovirus protein, or a herpes simplex virus protein.
Embodiment 5 includes the method of any one of Embodiments 1 to 3, wherein the virus protein is an AAV capsid protein.
Embodiment 6 includes the method of any one of Embodiments 1 to 3, wherein the virus protein is an adenovirus protein.
Embodiment 7 includes the method of Embodiment 6, wherein the adenovirus protein is an adenovirus 5, 26, 35 or 48 protein.
Embodiment 8 includes the method of Embodiment 6, wherein the adenovirus protein is adenovirus 5 protein.
Embodiment 9 includes the method of any one of Embodiment 1 or Embodiment 2, wherein the virus protein is a lentivirus protein.
Embodiment 10 includes the method of any one of Embodiments 1 to 9, wherein the virus protein is dissolved in a mixture comprising SDC at about 0.01% to 1.5% (w/w) and DDM at about 0.01% to 1.0% (w/w).
Embodiment 11 includes the method of Embodiment 10, wherein the virus protein is dissolved in a mixture comprising SDC at about 0.5% to 1.5% (w/w) and DDM at about 0.01% to 1.0% (w/w).
Embodiment 12 includes the method of Embodiment 12, wherein the virus protein is dissolved in a mixture comprising SDC at about 0.5% to 1.5% (w/w) and DDM at about 0.2% to 1.0% (w/w).
Embodiment 13 includes the method of Embodiment 12, wherein the mixture comprises SDC at about 0.75% to 1.25% (w/w) and DDM at about 0.5% to 0.8% (w/w).
Embodiment 14 includes the method of Embodiment 10, wherein the mixture comprises SDC at about 0.01% to 0.6% (w/w) and DDM at about 0.01% to 1.0% (w/w).
Embodiment 15 includes the method of Embodiment 14, wherein the mixture comprises SDC at about 0.01% to 0.6% (w/w) and DDM at about 0.01% to 0.6% (w/w).
Embodiment 16 includes the method of Embodiment 15, wherein the mixture comprises SDC at about 0.2% to 0.4% (w/w) and DDM at about 0.05% to 0.2% (w/w).
Embodiment 17 includes the method of any one of Embodiments 1 to 3, wherein the mixture comprises a ratio of about 1:0.5 w/w or about 3.5:1 w/w (SDC: DDM).
Embodiment 18 includes the method of any one of Embodiments 1 to 17, wherein the dissolving in a solution occurs at about pH 6.0 to about pH 9.0.
Embodiment 19 includes the method of any one of Embodiments 1 to 18, wherein the digesting takes place at about 30° C. to 40° C., for a period of about 2 to 12 hours.
Embodiment 20 includes the method of any one of Embodiments 1 to 19, wherein the precipitating comprises precipitation with chloroform/methanol/water and centrifugation.
Embodiment 21 includes the method of any one of Embodiments 1 to 20, wherein the digesting comprises digesting with trypsin.
Embodiment 22 includes the method of Embodiment 21, wherein the digesting is done at a ratio of about 20:1 to about 100:1 w:w of virus protein: trypsin.
Embodiment 23 includes the method of any one of Embodiments 1 to 22, wherein the digested virus protein is about 3 to 70 amino acids in length.
Embodiment 24 includes the method of any one of Embodiments 2 to 23, wherein the analyzing comprises injecting the digested virus protein into a Liquid Chromatography

Mass Spectrometer, without first performing a buffer exchange or a desalting step.
Embodiment 25 includes the method of any one of Embodiments 2 to 24, wherein a solution volume that is analyzed by LC-MS/MS is less than 50 μL.
Embodiment 26 includes the method of any one of Embodiments 1 to 25, wherein the sample containing the virus protein has a concentration of virus protein of about 0.001 mg/mL to about 0.10 mg/mL.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

1. A method of preparing a digested virus protein, comprising:
a. precipitating a virus protein from a sample containing the virus protein;
b. dissolving the virus protein in a mixture comprising sodium deoxycholate (SDC) and N-dodecyl-beta-D-Maltoside (DDM) to generate a solution; and
c. digesting the virus protein with a protease.

2. A method of analyzing a digested virus protein, comprising:
a. precipitating a virus protein from a sample containing the virus protein;
b. dissolving the virus protein in a mixture comprising sodium deoxycholate (SDC) and N-dodecyl-beta-D-Maltoside (DDM) to generate a solution;
c. digesting the virus protein with a protease; and
e. analyzing the digested virus protein via liquid chromatography-tandem mass spectrometry (LC-MS/MS).

3. The method of claim 2, further comprising the step of:
d. removing the SDC from the solution;
wherein step d is performed after step c) and before step e).

4. The method of claim 1, wherein the virus protein is an adeno-associated virus capsid protein (AAV capsid protein), an adenovirus protein, a lentivirus protein, a retrovirus protein, or a herpes simplex virus protein.

5. The method of claim 1, wherein the virus protein is an AAV capsid protein.

6. The method of claim 1, wherein the virus protein is an adenovirus protein.

7. The method of claim 6, wherein the adenovirus protein is an adenovirus 5, 26, 35 or 48 protein.

8. The method of claim 6, wherein the adenovirus protein is adenovirus 5 protein.

9. The method of claim 1, wherein the virus protein is a lentivirus protein.

10. The method of claim 1, wherein the virus protein is dissolved in a mixture comprising SDC at about 0.01% to 1.5% (w/w) and DDM at about 0.01% to 1.0% (w/w).

11. The method of claim 10, wherein the virus protein is dissolved in a mixture comprising SDC at about 0.5% to 1.5% (w/w) and DDM at about 0.01% to 1.0% (w/w).

12. The method of claim 1, wherein the virus protein is dissolved in a mixture comprising 1) SDC at about 0.5% to 1.5% (w/w) and DDM at about 0.2% to 1.0% (w/w), 2) SDC at about 0.75% to 1.25% (w/w) and DDM at about 0.5% to 0.8% (w/w), 3) SDC at about 0.01% to 0.6% (w/w) and DDM at about 0.01% to 1% (w/w), 4) SDC at about 0.01% to 0.6% (w/w) and DDM at about 0.01% to 0.6% (w/w) or 5) wherein the mixture comprises SDC at about 0.2% to 0.4% (w/w) and DDM at about 0.05% to 0.2% (w/w).

13. (canceled)

14. (canceled)

15. (canceled)

16. (canceled)

17. The method of claim 10, wherein the mixture comprises a ratio of about 1:0.5 w/w or about 3.5:1 w/w (SDC: DDM).

18. The method of claim 1, wherein step b. of dissolving in a solution occurs at about pH 6.0 to about pH 9.0.

19. The method of claim 1, wherein step c. of digesting takes place at about 30° C. to 40° C., for a period of about 2 to 12 hours.

20. The method of claim 1, wherein step a. of precipitating comprises precipitation with chloroform/methanol/water and centrifugation.

21. The method of claim 1, wherein step c. of digesting comprises digesting with trypsin, optionally wherein the digesting is done at a ratio of about 20:1 to about 100:1 w:w of virus protein: trypsin.

22. (canceled)

23. The method of claim 1, wherein the digested virus protein is about 3 to 70 amino acids in length.

24. The method of claim 1, wherein step e. of analyzing comprises injecting the digested virus protein into a Liquid Chromatography Mass Spectrometer, without first performing a buffer exchange or a desalting step, optionally wherein a solution volume that is analyzed by LC-MS/MS is less than 50 μL.

25. (canceled)

26. The method of claim 1, wherein the sample containing the virus protein has a concentration of virus protein of about 0.001 mg/mL to about 0.10 mg/mL
